(19) United States
(12) Patent Application Publication
(10) Pub. No.: US 2021/0225513 A1
(43) Pub. Date: Jul. 22, 2021

(54) Method to Create Digital Twins and use the Same for Causal Associations

(71) Applicant: XY.Health Inc., Cambridge, MA (US)

(72) Inventors: Arjun K. MANRAI, North Easton, MA (US); Chirag J. PATEL, Boston, MA (US)

(73) Assignee: XY.Health Inc., Cambridge, MA (US)

(21) Appl. No.: 17/156,499

(22) Filed: Jan. 22, 2021

Related U.S. Application Data
(60) Provisional application No. 62/964,133, filed on Jan. 22, 2020.

(51) Int. Cl. G16H 50/20; G16H 50/70; G16H 10/20; G16H 10/60

(52) U.S. Cl. CPC G16H 50/20; G16H 10/60; G16H 10/20; G16H 50/70

(57) ABSTRACT

The technology disclosed relates to systems and methods for predicting digital twins. The system includes logic to use a machine learning model predict correlation between pairs of persons and save the results in an environmental and phenotypic correlation matrix. The inputs to the machine learning model can include data from individual-level and group-level datasets. The individual-level datasets include administration dataset including clinical data and person dataset including personal data. The group-level datasets include exposome dataset including environmental exposure and subpopulation dataset. The system includes logic to use the environmental and phenotypic correlation matrix as a random effect when determining associations between exposures and outcomes. The system includes a second machine learning model that can take a pair of exposure and outcome and the environmental and phenotypic correlation matrix as input to predict causal association between exposure and outcome.

### PRIORITY APPLICATION

This application claims the benefit of U.S. Patent Application No. 62/964,133, entitled “METHOD TO CREATE DIGITAL TWINS AND USE THE SAME FOR CAUSAL ASSOCIATIONS,” filed Jan. 22, 2020 (Attorney Docket No. XYAI 1001-1). The provisional application is incorporated by reference for all purposes.

### FIELD OF THE TECHNOLOGY DISCLOSED

The technology disclosed relates to use of machine learning techniques to process individual and group-level data to predict digital twins.

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

In the field of medical research and treatment, the gold standard for determining whether an intervention causes a desired effect, either at individual or population level, is randomized experiment. As a traditional and standard way, when making everyday clinical care decisions for an individual patient with chronic disease, such as altering the medicine regimen for type II diabetes, it is desired as ideal to have well-powered and randomized controlled trials (RCTs) consisting of subjects that adequately model the individual patient. Such trials are expensive in cost and onerous in effort, and often lacking for most clinical care decisions. A large amount of data is available from existing datasets collected for epidemiological, administrative (such as insurance claims) or other purposes. Determining causal relationships from such datasets is challenging. For example, one challenge is the presence of confounding factors that impact both exposures and outcomes thus impacting the causal relationships. It is difficult to identify various confounding factors when using existing datasets.

Therefore, an opportunity arises to develop a system that can predict causal relationships between exposures and outcomes from existing datasets.

### BRIEF DESCRIPTION OF THE DRAWINGS

FIG. 1 is a high-level architecture of a system that can be used to predict digital twins and determine causal relationship between exposures and outcomes.

FIG. 2 is an example environmental and phenotypic relatedness matrix that can be used to determine distance between pairs of persons.

FIG. 3 is an example digital twins pipeline integrating multiple existing datasets to identify digital twins.

FIG. 4 illustrates training a machine learning model to predict digital twins.

FIG. 5 illustrates generation of a correlation matrix using a trained machine learning model.

FIG. 6A illustrates using a machine learning model to predict causal associations between exposures and outcomes using digital twins as an additional input.

FIG. 6B illustrates a high-level workflow to derive and verify causal association utilizing digital twins.

FIG. 7 is a flow chart illustrating an example workflow to derive and verify causal association using digital twins.

FIG. 8 is an example of integrating data from multiple datasets.

FIG. 9 is a simplified block diagram of a computer system that can be used to implement the technology disclosed.

FIG. 10 is an example convolutional neural network (CNN).

FIG. 11 is a block diagram illustrating training of the convolutional neural network of FIG. 10.

### DETAILED DESCRIPTION

The following discussion is presented to enable any person skilled in the art to make and use the technology disclosed, and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown, but is to be accorded the widest scope consistent with the principles and features disclosed herein.

### INTRODUCTION

In the field of medical research and treatment, the gold standard for determining whether an intervention causes a desired effect, either at individual or population level, is randomized experiment. As a traditional and standard way, when making everyday clinical care decisions for an individual patient with chronic disease, such as altering the medicine regimen for type II diabetes, it is ideal to have well-powered and randomized controlled trials (RCTs) consisting of subjects that adequately model the individual patient. Such trials are expensive in cost and onerous in effort, and often lacking for most clinical care decisions.

Data from observational studies can be helpful to determine causal effects in health care research. Many observational and retrospective big datasets are available today, often gathered for epidemiological and/or administrative purposes, e.g., insurance claims, electronic health records, laboratory reports, or data collected from medical and wearable devices, etc. Estimates made from observations are correlational, i.e., how factor X (such as an exposure) is correlated with factor Y (such as an outcome). While a prerequisite for causal relationship, correlation is not equal to causation. Undoubtedly, observational data can be combined and analyzed computationally to estimate the causal effect of an intervention or a risk factor.

However, such interference of the causal effect is full of challenges. One of the challenges is fully addressing confounding, i.e., the existence of a variable that is related to both exposures (e.g., dietary intake, smoking, etc.) and outcomes (e.g., obesity, lung cancer, etc.). Confounding arises due to a mismatch between individuals i.e., the one who receives an intervention and the other who gets the disease. Another challenge is reverse causality. Reverse causality occurs when individuals receive an intervention during the trajectory of the outcome, challenging the temporal relationship between the intervention and the outcome. Therefore, to address above challenges, a perfectly matched individual is desired. A perfectly matched individual is a person's exact twin, that out of the person and his/her twin, one receives an intervention and the other does not. This way, all experiences including behavioral, genetic, and environmental factors are all considered.

Propensity score can also be derived from large prior collected large datasets and used to estimate causal effects from such datasets. Propensity score is the conditional probability of assignment to a particular treatment given a vector of observed covariates. Also, case-crossover studies have been demonstrated to be efficient provided that the case-crossover study is carefully designed and carefully controlled. Scientists and researchers have applied the method into large scale quantitative analysis. Moreover, in recent years, systematic searches for exposome associations based on massive X-Y testing have been explored. The technology disclosed presents a digital pipeline to integrate retrospective data and utilize that data to determine digital twins.

An alternative method is to perform randomized control trials to evaluate individual and population level decisions. However, this method is extremely expensive and onerous, and many times ethically impossible due to potential harmful exposure which cannot be randomized. It is also not scalable to investigate multiple exposures in a database and very difficult to recruit patient populations at risk for a certain disease.

The technology disclosed presents systems and methods for creation of digital twins and further utilizing the created digital twins for estimations of causal association between factors (or exposures) and outcomes for the applications of medical and healthcare planning.

The method to create digital twins in the field of healthcare applications includes defining a matrix comprising a plurality of phenotypic and environmental factors, measuring distance between any two individuals' data in a data cohort based on values of the plurality of phenotypic and environmental factors of each individual. The technology disclosed can use a machine learning model to identify the most likely phenotypic twins with the lowest value of distance measured. The method can include identifying phenotypic twins with the lowest value of distance between them as digital twins.

The technology disclosed provides a method to estimate causal association using digital twins. The method comprises integrating and cross-referencing data of a plurality of databases, joining the integrated data of a plurality of databases with personal information, categorizing the joined data of a plurality of databases with personal information into one or more exposure variables and one or more outcome variables. The method includes creating digital twins, identifying one or more causal associations between the one or more exposure variables and the one or more outcome variables, estimating robustness of the identified causal association between the one or more exposure variables and the one or more outcome variables, and output the one or more causal associations.

Digital twins creation and causal association formation methods can be applied for hospitals to predict medical health care utilization. Digital twins creation and causal association formation methods can be applied for estimation of causes and effect of diseases and trends for users such as major health insurance companies and hospitals. Such system comprises a server such as a web application program to display predictions to users. Such system can be configured to enable users to query aggregate health statistics and cause and effect trends for certain regions and individuals. Digital twins creation and causal association formation methods can be applied to predict and manage risk and uncertainty in actuarial processes. Health, housing, and life insurances require biomedical data for accurate assessment of risk to optimize pricing of insurance instruments. Digital twins creation and causal association formation methods can be also applied to present risk at personal level. Individual disease risk as a function of biomedical factors estimated as a probability can be displayed to end users.

### Environment

Many alternative embodiments of the present aspects may be appropriate and are contemplated, including as described in these detailed embodiments, though also including alternatives that may not be expressly shown or described herein but as obvious variants or obviously contemplated according to one of ordinary skill based on reviewing the totality of this disclosure in combination with other available information. For example, it is contemplated that features shown and described with respect to one or more embodiments may also be included in combination with another embodiment even though not expressly shown and described in that specific combination.

For purposes of efficiency, reference numbers may be repeated between figures where they are intended to represent similar features between otherwise varied embodiments, though those features may also incorporate certain differences between embodiments if and to the extent specified as such or otherwise apparent to one of ordinary skill, such as differences clearly shown between them in the respective figures.

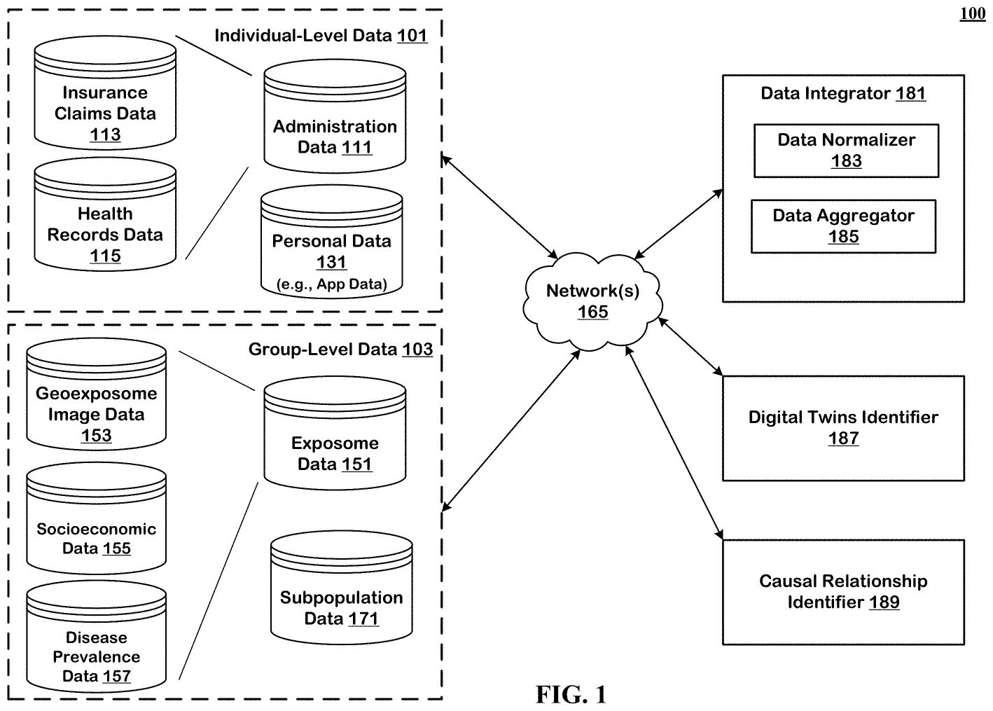

We describe a system for predicting digital twins and further using the digital twins to reduce or eliminate the impact of confounding factors when predicting causal relationships between exposures and outcomes. The system is described with reference to FIG. 1 showing an architectural-level schematic 100 of a system in accordance with an implementation. Because FIG. 1 is an architectural diagram, certain details are intentionally omitted to improve the clarity of the description. The discussion of FIG. 1 is organized as follows. First, the elements of the figure are described, followed by their interconnection. Then, the use of the elements in the system is described in greater detail.

FIG. 1 includes the system 100. This paragraph names labeled parts of system 100. The system includes a plurality of databases including an individual-level database 101, a group-level database 103, a data integrator 181, a digital twins identifier 187, and a causal relationship identifier 189. The data integrator 181 can comprise a data normalizer 183 and a data aggregator 185.

The individual-level database 101 can comprise an administration database 111 and a personal database 131. The administration database 111 can comprise an insurance claims database 113, and a health records database 115. The group-level database 103 can comprise an exposome database 151 and a subpopulation database 171. The exposome database 151 can comprise a geoexposome image database 153, a socioeconomic database 155, and a disease prevalence database 157. The system 100 can also include other databases to store data collected from previously conducted clinical trials, observational studies, publicly available data, proprietary or private data, etc.

As used herein, no distinction is intended between whether a database is disposed “on” or “in” a computer readable medium. Additionally, as used herein, the term “database” does not necessarily imply any unity of structure. For example, two or more separate databases, when considered together, still constitute a “database” as that term is used herein.

The processing engines in system 100, including data integrator 181, digital twins identifier 187, and causal relationship identifier 189 can be deployed on one or more network nodes connected to the network(s) 165. Also, the processing engines described herein can execute using more than one network node in a distributed architecture. As used herein, a network node is an addressable hardware device or virtual device that is attached to a network, and is capable of sending, receiving, or forwarding information over a communications channel to or from other network nodes. Examples of electronic devices which can be deployed as hardware network nodes include all varieties of computers, workstations, laptop computers, handheld computers, and smartphones. Network nodes can be implemented in a cloud-based server system. More than one virtual device configured as a network node can be implemented using a single physical device.

A network(s) 165 couples the data integrator 181, the digital twins identifier 187, the causal relationship identifier 189, the individual-level database 101 and the group-level database 103.

The data integrator 181 can include logic to integrate data from various databases for use as input to machine learning models. The digital twins identifier 187 can include logic to determine a correlation value for a first person (or a subject, patient, etc.) that indicates a distance of the first person with a second person in the plurality of persons in the population or dataset under analysis. The digital twins identifier can use inputs from one or more databases listed above. The digital twins identifier can include a machine learning model (such as a regressor). The trained machine learning model can be deployed to predict digital twins. The digital twins identifier can include logic to output a correlation value using trained machine learning model, indicating distance between the first person and the second person and compare the correlation value with a threshold to determine whether the second person is a digital twin of the first person. In one implementation, the correlation values can range between 0 and 1. If the correlation value is above a threshold, e.g., 0.6 then the second person can be predicted as a digital twin of the first person. The threshold can be set at a higher level or at a lower level than 0.6. The technology disclosed can produce an environmental and phenotypic correlation matrix containing correlation values between pairs of persons in the dataset.

The technology disclosed can determine a ranked list of causal relationships between a plurality of exposures and a plurality of outcomes. As described above, when we use data from observational datasets, confounding can cause problems when identifying causal relationships between exposures and outcomes. The technology disclosed includes logic to reduce the impact of confounding factors when determining the association between the exposures and outcomes. The causal relationship identifier 189 include the logic to provide the environmental and phenotypic correlation matrix as an additional input to the machine learning model as a “random effect” to control the environmental relatedness between individuals. The causal relationship identifier 189 can systematically iterate for each exposure in the plurality of exposures and determine the association between that exposure and each outcome in the plurality of outcomes. The machine learning model can predict an association value e.g., in a range between 0 and 1. The results of the associations between pairs of outcomes and exposures can be stored in an X-Y association matrix. In the following section, we present further details of the environmental and phenotypic relatedness matrix.

Completing the description of FIG. 1, the components of the system 100, described above, are all coupled in communication with the network(s) 165. The actual communication path can be point-to-point over public and/or private networks. The communications can occur over a variety of networks, e.g., private networks, VPN, MPLS circuit, or Internet, and can use appropriate application programming interfaces (APIs) and data interchange formats, e.g., Representational State Transfer (REST), JavaScript Object Notation (JSON), Extensible Markup Language (XML), Simple Object Access Protocol (SOAP), Java Message Service (JMS), and/or Java Platform Module System. All of the communications can be encrypted. The communication is generally over a network such as the LAN (local area network), WAN (wide area network), telephone network (Public Switched Telephone Network (PSTN), Session Initiation Protocol (SIP), wireless network, point-to-point network, star network, token ring network, hub network, Internet, inclusive of the mobile Internet, via protocols such as EDGE, 3G, 4G LTE, Wi-Fi and WiMAX. The engines or system components of FIG. 1 are implemented by software running on varying types of computing devices. Example devices are a workstation, a server, a computing cluster, a blade server, and a server farm. Additionally, a variety of authorization and authentication techniques, such as username/password, Open Authorization (OAuth), Kerberos, Secured, digital certificates and more, can be used to secure the communications.

### Environmental and Phenotypic Relatedness Matrix

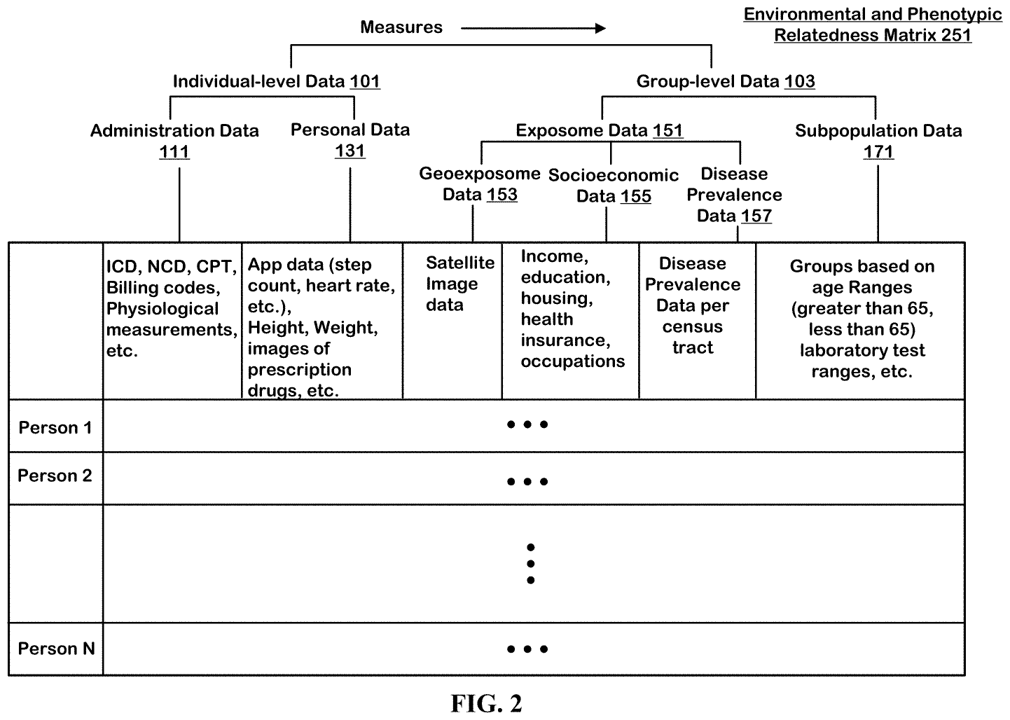

FIG. 2 presents an example environmental and phenotypic relatedness matrix 251. The matrix can be used to store records for persons. The records can include measurements, images or other types of data obtained from a variety of databases as described above. The data related to a person is stored as a row in the environmental and phenotypic relatedness matrix. The matrix can have up to N rows corresponding to N persons in the population. The data in the environmental and phenotypic relatedness matrix can represent different types of observational datasets organized in different databases as illustrated in FIG. 1. The data can be linked across datasets using a person's person-level identifier or group-level identifier. Person-level identifiers (such as name, patient identifier, social security number (SSN), etc.) can identify data related to a specific person. Group-level identifiers can identify group-level data for a person such as census tract-level data or subpopulation data. Person attributes such as address, age, gender, etc. can be used to select data from group-level datasets such as census tract-level data or range-bound data such as laboratory ranges, age-ranges etc. As shown in FIG. 2, the measures (recorded as columns) in the matrix are organized according to individual-level database 101 and group-level database 103.

FIG. 2 presents an example environmental and phenotypic relatedness matrix for illustration purpose. The environmental and phenotypic relatedness matrix can include data from additional databases not shown in FIG. 2. The individual-level database 101 can comprise of administration database 111 and personal database 131. Group-level database 103 can comprise exposome database 151 and subpopulation database 171. The exposome database 151 further comprises the geoexposome database 153, the socioeconomic database 155, and the disease prevalence database 157. We now present examples of data that can be used from different types of observational datasets organized in the databases listed above. It is understood that these datasets are presented as examples to illustrate the technology disclosed. The system can use additional datasets from public or proprietary sources. In the following section, we present details of different types of datasets.

### Examples of Observational Datasets

We have organized the various datasets under two high-level categories: individual-level database 101 and group-level database 103.

Individual-Level Database

Individual-level database comprises data related to persons (or subjects) from health records, medical devices, personal devices, or wearable devices. The individual-level data can be organized into two databases i.e., administration database 111 and personal database 131.

Administration Database

Administrative data are the central source of information on the health status of an individual (or person) as recorded by insurance companies, hospitals (electronic health record), and/or epidemiological cohorts. These data can also include laboratory and physiological measurements, disease history or billing codes. Information from these sources can be mapped to various coding systems, including the International Classification of Diseases (ICD), National Drug Codes (NDC), Current Procedural Terminology (CPT), Logical Observation Identifiers Names and Codes (LOINC), Systematized Nomenclature of Medicine Clinical Terms (SNOMED), and others. Personal data can also contain disease codes and other patient-level attributes that can identify phenotypic relatedness between persons.

Personal Database

Personal data can be collected from personal devices, health tracking devices, medical devices, mobile devices, wearable devices, etc. The personal data can be collected from integration with health mobile apps e.g., APPLE RESEARCHKIT™, apps deployed specifically for the digital twins system, or from other organizations such as contract research organizations (CROs). This can be a potential point of recruitment and consent for individuals and provides information about individuals not available in the administrative data. The data in personal database can include passively recorded information (such as location, step count from personal devices) or actively recorded information (patient provided on the interface of the app).

Group-Level Database

Group-level database comprises data related to groups or subpopulations of persons (or subjects). Group-level data can also be referred to as aggregate data. Group-level data can comprise two types of databases, i.e., exposome database 151 or subpopulation database 171. Exposome database stores records related to various types of exposures related to groups or subpopulations of persons. Exposome database 171 can comprise of geoexposome image data 153, socioeconomic database 155 (also referred to as demographic and socioeconomic database) and disease prevalence database 157.

Geoexposome Image Database

Geoexposome image database 153 can contain satellite image data of built environment. The built environment can indicate roads, parks, walking paths, and different types of buildings such as schools, hospitals, libraries, sports arenas, residential and commercial areas in a community, neighborhood, or a city, etc. The image data can be organized at census tract-level or aggregated to a city-level. Millions of satellite images (e.g., n=4,742,919) are analyzed using an unsupervised deep learning algorithm and a supervised machine learning algorithm. Images can be extracted in tiles from the different data sources such as OpenMapTiles database using the coordinate geometries of the census tracts. After extraction, images can be digitally enlarged to achieve a zoom level of 18.

In one implementation, the satellite image data can be encoded with temporal information such as timestamps. The geotemporal data includes time series metrics for a plurality of environmental conditions over a time period. Environmental conditions can include pollution related data collected from sensors per unit of time such as hourly, daily, weekly, or monthly, etc. The geotemporal data includes time series metrics for a plurality of climate conditions over a time period. Climate conditions can indicate weather conditions such as cold, warm, etc. The climate related data can be collected from sensors over a period of time such as hourly, daily, weekly, or monthly, etc.

Examples of different types of satellite image data that can be stored in the geoexposome image database and used by the technology disclosed are presented below.

OpenMapTiles

The images are satellite raster tiles that are downloaded from the OpenMapTiles (available at openmaptiles.com) database (n=4,742,919). The images can have a spatial resolution close to 20 meters per pixel. Images can be extracted in tiles from the OpenMapTiles database using the coordinate geometries of the census tracts. After extraction, images are digitally enlarged to achieve a zoom level of 18.

PlanetScope

The PlanetScope images (available at planet.com/products/planet-imagery/) from Planet Labs2 are raster images which have been extracted in a way such that we have complete geometry extractions of the desired census tract. These raster images are extracted in the GeoTIFF format and have a spatial resolution between 3 meters/pixel to 5 meters/pixel which is resampled to provide a 3 meters/pixel resolution thereby allowing a zoom level of somewhere between 13 and 15. Once the geometries are extracted, the images are broken down into tiles for the digital twins pipeline.

SkySat

The SkySat images (available at planet.com) is another product of Planet Labs which has the highest spatial resolution out of all of its products. Similar to PlanetScope images, the SkySat images are complete geometry extractions of the desired census tract. The raster images are extracted in a GeoTIFF format and have a spatial resolution of about 0.72 meters/pixel which is then resampled to 0.5 meters/pixel and thus allowing a zoom level somewhere between 16 and 18. Once the geometries are extracted, the images can be broken down into tiles for processing by the digital twins pipeline.

Socioeconomic Database

Socioeconomic database can include socioeconomic and demographic data from 5-year 2013-2017 American Community Survey (ACS) Census. The ACS census data can contain sociodemographic prevalences and median values for census tracts. Examples of data can include the following:
Total population
Area in square kilometers
Ethnicity: White percentage, Black percentage, Native American percentage, Hawaiian-Pacific Islander percentage, Other ethnicity percentage, two or more races percentage, two races excluding some Other race & three or more races, two races including some Other race, Hispanic percentage
Income indicators: Median household income, population below poverty line, public assistance income within last 12 months, median home value, public assistance income, Gini index, unemployment rate, population percentage under 100 percent of poverty line, population percentage from 100-150% of poverty line, population percentage from 150-200% of poverty line.
Education Indicators: College graduate percentage, no high school diploma percentage
Housing Indicators: >1 occupant per room percentage, >1.5 occupants per room percentage, >2 occupants per room percentage, median year house built, lacking plumbing facilities percentage, household 2+, household 3+, household 4+, household 5+, household 6+, household 7+
Health Insurance Type: Private insurance percentage, Medicare insurance percentage, Medicaid insurance percentage, military/VA insurance percentage, private & Medicare insurance percentage, Medicare & Medicaid insurance percentage.
Age: Over age 65 (all), over age 65 (male), over age 65 (female), under age 19 (all), under age 19 (male), under age 19 (female)
Occupation: management/financial business, computer engineering/science, legal, community/social service, education/training/library, healthcare practitioner, healthcare support, protective services, food preparation services, cleaning/maintenance, personal care & service, sales office, natural resource construction/maintenance, production/transportation material moving, commute via public transportation percentage, commute via vehicle percentage, commute via walking percentage, work from home percentage.

In one implementation, the socioeconomic data can be encoded with temporal data. The temporal data can include time series metrics for changes to a plurality of sociodemographic variables over a time period. For example, it can indicate changes in median income of population in a geographic area such as a census tract on a per yearly basis. The frequency of data collection can change without impacting the processing performed by the digital twins pipeline. In another implementation, this socioeconomic data can be integrated with geoexposome data to form geotemporal data. The geotemporal data can then use used in the environmental and phenotypic relatedness matrix 251.

Disease Prevalence Database

The disease prevalence and risk factors data can be sourced from the US Centers for Disease Control and Prevention 2017 500 Cities data. The 500 Cities data contains disease and health indicator prevalence for 26,968 individual census tracts of the 500 Cities which are the most populous in the United States. These prevalences are estimated from the Behavioral Risk Factor Surveillance System. Examples of fields for which data can be stored in this database include Arthritis, asthma, hypertension, cancer, high cholesterol, kidney disease, chronic obstructive pulmonary disease (COPD), coronary heart disease (CHD), diabetes, mental health not good for >=14 days, physical health not good for >=14 days, all teeth lost, stroke, lack of health insurance in population aged 18-64, routine checkup within past year, dental visit within past year, blood pressure medication, cholesterol screening, mammography use, pap smear use, colon screen, up-to-date on core preventative services for male population aged >=65, up-to-date on core preventative services for female population aged >=65, binge drinking, smoking, obesity, no leisure-time physical activities, sleep <7 hours, median household income, population, population density.

Subpopulation Database

Subpopulation database 157 can include data that is integrated together on the basis of subpopulation information, such as an age range, laboratory range, gender, ethnicity or some other characteristic that defines a group. For example, the technology disclosed can extract information from clinical practice guidelines and organize the data according to subgroups based on age, gender, ethnicity, etc. We present example of such data in Table 1. The hypertension clinical practice guidelines are available at <ahajournals.org/doi/10.1161/HYPERTENSIONAHA.120.15026> and the diabetes clinical practice guidelines are available at <pro.aace.com/disease-state-resources/diabetes/clinical-practice-guidelines-treatment-algorithms/comprehensive>. The values in demographic information column in Table 1 can be used to form subpopulations and codes for different subpopulations can be used in the environmental and phenotypic relatedness matrix.

TABLE 1
Example of Information Extracted
from Clinical Practice Guidelines

| | Demographic | | |
|---|---|---|---|
| Guideline | Information | ICD Codes | NDC Codes |
| Hypertension | Age > . . . | I10 Essential | 0172-2083-60 - |
| Clinical | Gender = . . . | Hypertension | Hydrochlorothiazide |
| Practice | Ethnicity = . . . | I11 Hypertension & | 0172-2083-80 - |
| Guidelines | | Heart Disease | Hydrochlorothiazide |
| | | . . . | . . . |
| Diabetes | Age > . . . | E08 Diabetes due to | 62037-571-01 - |
| Clinical | Gender = . . . | underlying condition | Metformin |
| Practice | Ethnicity = . . . | E08.00 Diabetes due | 62037-571-10 - |
| Guidelines | | to NKHHC | Metformin |
| | | . . . | 62037-577-01 - |
| | | | Metformin |
| | | | 62037-577-10 - |
| | | | Metformin |
| | | | . . . |

### Digital Twins Pipeline

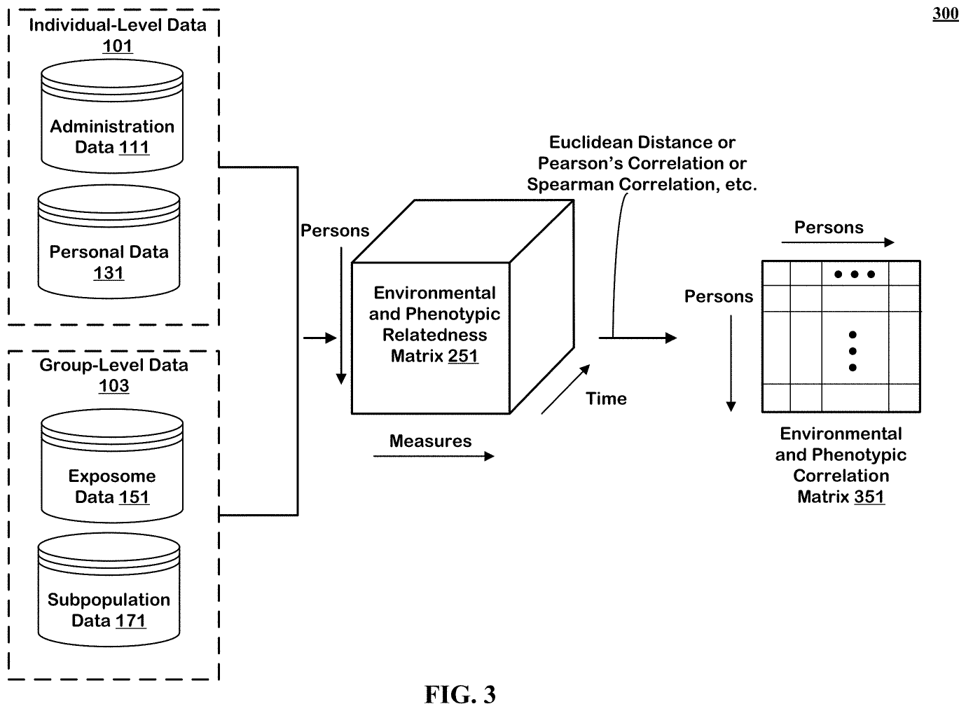

FIG. 3 presents an example digital twins pipeline 300 that includes integrating the various datasets from individual-level and group-level databases. The technology disclosed can pre-process some of data before calculating distance between persons.

Preprocessing

We present some example preprocessing of data for illustration purposes. The satellite images from the geoexposome database 153 can be passed through AlexNet, a pretrained convolutional neural network (CNN), in an unsupervised deep learning approach called feature extraction. The resulting vector from this process is a “latent space feature” representation of the image comprising 4,096 features. This latent space representation is an encoded (non-human readable) version of the visual patterns found in the satellite images, which, when coupled with machine learning approaches, is used to model the built environment of a given census tract. For each census tract, we calculate the mean of the latent space feature representation.

For the other databases such as the ACS socioeconomic and demographic data, disease prevalence data we can calculate the weighted average by population to aggregate the data from the census tract level to the city level. Features from different data source can be standardized to mean 0 and unit variance. Similar pre-processing of individual-level data can be performed. The technology disclosed can also include observations over time thus making the environmental and phenotypic relatedness matrix a three-dimensional matrix as shown in FIG. 3. Each person can be considered as a vector with all attributes about that person (in matrix 251) and the distance between two vectors indicates their relatedness. The more distant the two vectors (large distance value) the less related they are to each other or less likely to be twin. The less distance between two vectors, the more likely they are to be twin. The system can use inputs from additional data sources such as genetic relatedness (e.g., sibling fraternal, or identical twin, or fraction of genetic relatedness). The system can also use distance between locations of two persons based on their location data when determining their relatedness.

We present three methods to determine digital twins. The first method determines digital twins between pairs of persons by calculating distance between vectors (or rows) representing persons in the environmental and phenotypic relationship matrix 251. The second method to determine digital twins is a non-linear approach using a machine learning model. The third method uses propensity scores matching.

First Method to Determine Digital Twins

We now refer to FIG. 3 to present the first method to determine digital twins by calculating distance between each pair of persons. The distance can be calculated using existing distance metrics such as Euclidean distance, Hamming distance, Pearson's correlation or Spearman rank-order correlation (Spearman correlation, for short). This results in an environmental and phenotypic correlation matrix 351 (also referred to as a correlation matrix) which is a N×N square matrix for a population size of N persons. The value in a cell of correlation matrix can indicate distance between the two persons (represented by the column and row values). If the value is zero or close to zero, the persons are not digital twins and if the value is one or close to one, the persons can be considered as twins (or digital twins). The system can use a threshold (such as 0.6) between zero and one so that when the correlation value is above the threshold, the persons are predicted as digital twins. If the correlation value is less than threshold than persons are not considered as digital twins. Threshold can be set at a higher value than 0.6 to only predict persons that have matching values for most of the input data.

Second Method to Determine Digital Twins

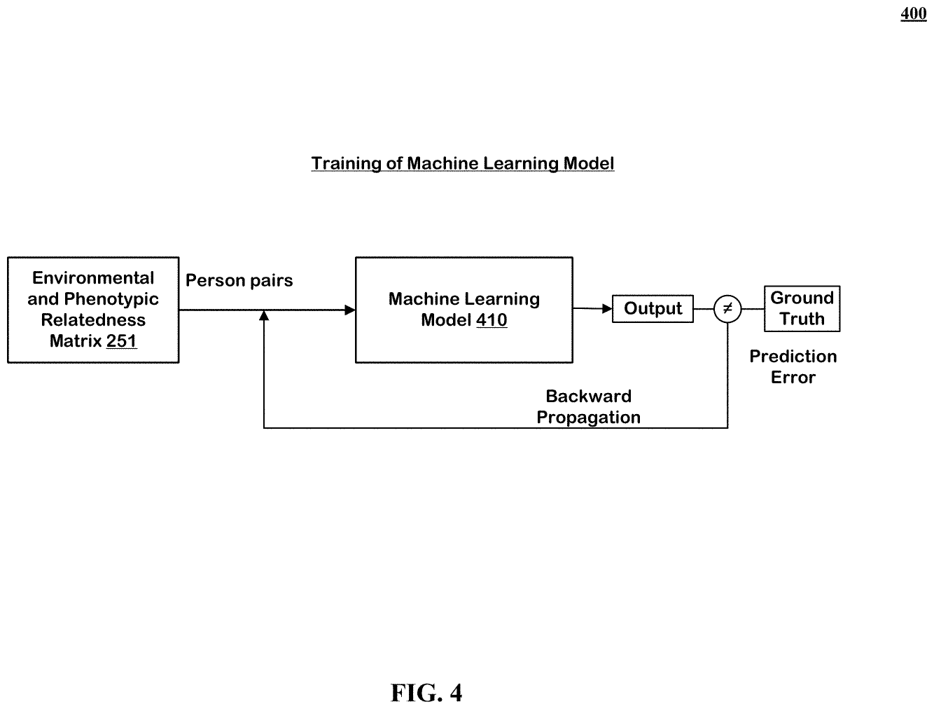

The second method to determine digital twins uses a trained machine learning model. FIG. 4 presents a high-level diagram 400 illustrating training a machine learning model 410 using the inputs from environmental and phenotypic relatedness matrix 251 as input. The training data can include labels to indicate the persons that are digital twins (or the ground truth values). In one example training process, the system can provide person pairs to the machine learning model 410. Thus, the input to machine learning model is two rows of the matrix 251 corresponding two persons in the person pair. The output from the machine learning model is a correlation value between 0 and 1. The output is compared with the ground truth value and prediction error is calculated. The model coefficients or weights are adjusted during backward propagation to reduce the prediction error so that they cause the output to be closer to the ground truth. A trained machine learning model is deployed to predict digital twins. The technology disclosed can use machine learning models such as LASSO (least absolute shrinkage and selection operator) which is a regression analysis method. Other type of regressors can be used such as extreme gradient boosting (XGBoost), multilayer perceptrons (MLPs), gradient boosted decision trees (GDBT), random forest, etc. Neural network models can also be used in digital twins pipeline.

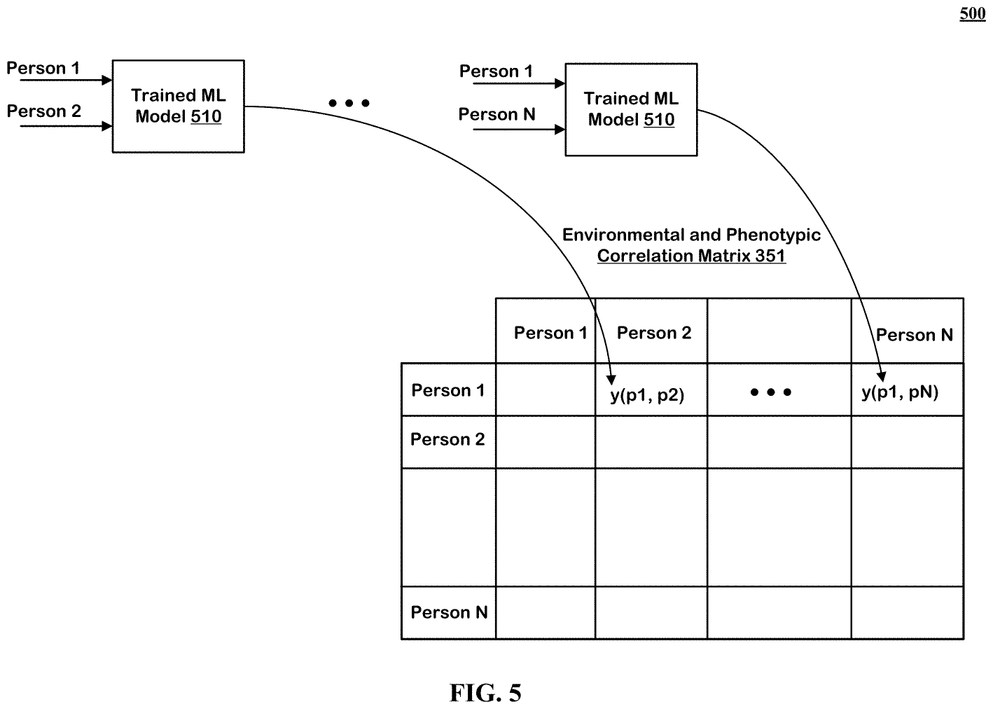

FIG. 5 (labeled 500) illustrates creation of environmental and phenotypic correlation matrix 351 using the trained machine learning model 510. The input to machine learning model is a pair of person records. The pair of person records can be taken from the environmental and phenotypic relatedness matrix 251. Therefore, each person input can be considered as a vector with values for all fields in the environmental and phenotypic relatedness matrix 251. For each person in the dataset (e.g., person 1) the machine learning model predicts a correlation value for every other person in the dataset. The correlation value can be between zero and one. FIG. 5 shows a correlation output y(p1, p2) for person 1 and person 2 and a correlation output y(p1, pN) for person 1 and person N, respectively, from the trained machine learning model 510. The trained machine learning model is used to fill correlation values for all pairs of persons under analysis.

Third Method to Determine Digital Twins

The third method uses propensity scores matching (PSM) to determine digital twins. PSM is a statistical matching technique that attempts to estimate the effect of a treatment, policy, or other intervention by accounting for the covariates that predict receiving the treatment. Propensity score tries to match individuals similar to determining digital twins but based on exposure or non-exposure. Suppose we want to understand or test association between smoking and lung cancer. Suppose X variable (or exposure) is smoking and Y variable (or outcome) is lung cancer. In the propensity score matching approach we find all persons in the dataset that smoke and all persons that do not smoke but the persons that smoke and that do not smoke are similar to each other on all other variables. For example, the persons in two groups (smoke vs do not smoke) may have same age, same sex, living in the same area, everything is the same except for smoking and not smoking. Propensity score indicates how similar the two persons are based on these characteristics. The propensity score matching method however, requires us to use a fixed number of exposures while the relatedness matrix approach presented in the first and second methods can use any number of inputs to determine digital twins. We now present how the technology disclosed uses the output from the digital twins pipeline to predict causal relationship by reducing or eliminating the impact of confounding factors.

### Predicting Causal (X-Y) Association Using Digital Twins

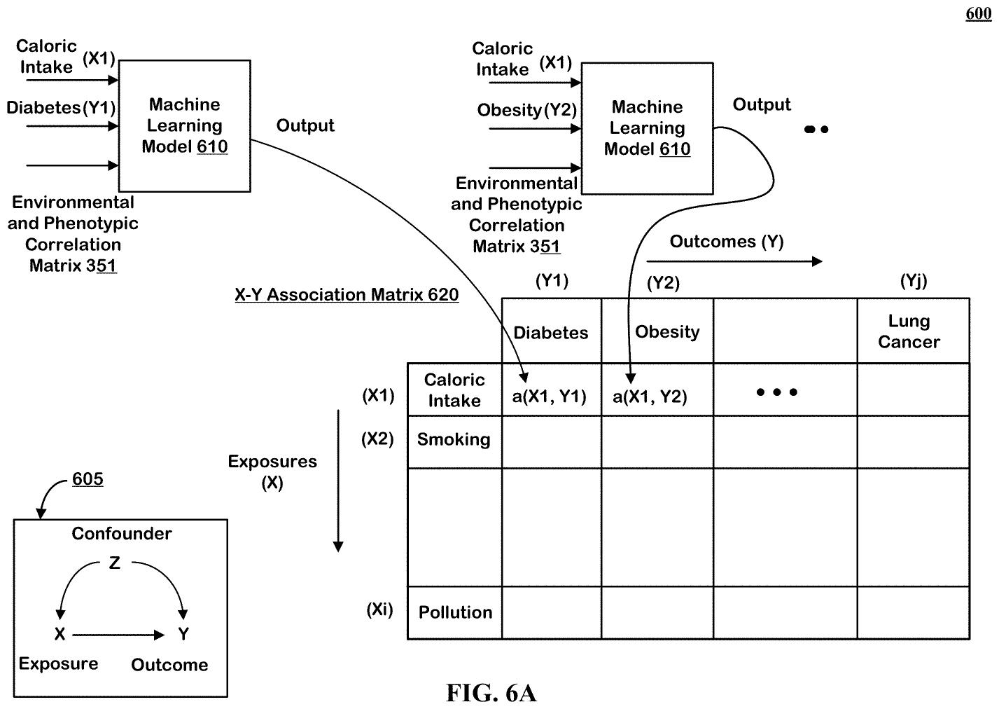

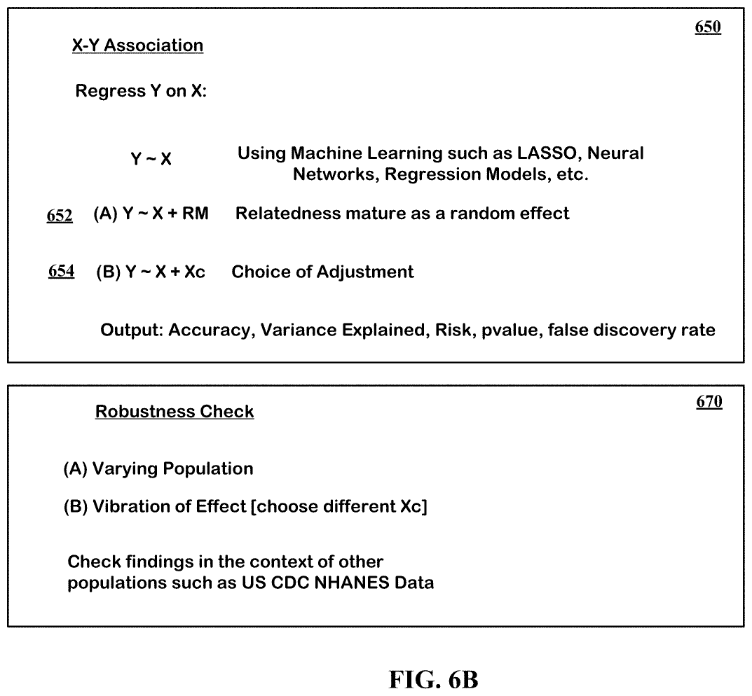

An issue faced by researchers when predicting causal relationship between exposures (X) and outcomes (Y) is confounding, especially when using observational datasets. Confounding is one of the three types of bias that may affect epidemiologic studies, the others being selection bias and information bias (misclassification and measurement error). Confounding is described as a confusion of effects. In other words, the effect of the exposure of interest (for example, caloric intake) on the outcome (for example, obesity) is confused with the effect of another risk or protective factor for the outcome (for example, diet pattern). The persons who have similar diet patterns could be confounding the relationship between the caloric intake and obesity. The confounding factor (referred to as Z) can impact both exposure (X) and outcome (Y) as shown in illustration 605 in FIG. 6A. To draw appropriate conclusions about the effect of an exposure (X) on an outcome (Y), we must separate its causal effect from that of the other factors (such as Z) that affect the outcome.

In the example described above, if we do not consider dietary patterns when determining the causal effect of caloric intake to obesity, the causal relationship may appear weak. For example, we know that socioeconomic factors can influence the diet patterns. The causal effect between caloric intake and obesity will appear strong if we assume or hypothesize that similar diet patterns are shared between individuals that have shared environment (or have similar socioeconomic factors). The technology disclosed can thus reduce the impact of confounding factors when determining causal relationships by providing environmental and the phenotypic correlation matrix 351 as input to the machine learning model 610. The technology disclosed uses the correlation matrix 351 as a way of adjusting for similarities, between persons, which can act as confounders to influence the association between exposures and outcomes. The output from the machine learning model 610 represents causal relationship (or association) between an exposure (X) and an outcome (Y) without the influence of confounding factors or with reduced influence of confounding factors.

An important feature of the technology disclosed is that it can reduce the impact of confounding factors between exposures and outcomes without the need for identifying the confounding factors for this purpose. The additional input (environmental and the phenotypic correlation matrix 351) provided to the machine learning model 610 acts as a random effect to reduce the impact of confounding factors on outcomes and exposures. Environmental and the phenotypic correlation matrix 351 indicates persons who are similar to each other (digital twins) and these persons share potential sources of confounding. The machine learning model thus adjusts its outputs to reduce the impact of confounding factors when determining an association between an exposure and an outcome. Therefore, the researchers do not need to know all the confounding factors prior to determining associations between exposures and outcomes. The technology disclosed reduces the impact of confounding factors when such analysis is performed using observational datasets.

The technology disclosed systematically predicts causal relationships between all pairs of exposures and outcomes as shown in illustration 600 in FIG. 6A. The exposures (X1 to Xi) are listed in rows of the X-Y association matrix 620. The outcomes (Y1 to Yj) are listed along the columns of the association matrix 620. The X-Y association values labeled as “a(Xi, Yj)” are listed in cells of the matrix. The association values can range from 0 to 1. The higher values represent a strong causal relationship between an exposure and outcome.

The system includes logic to train a separate machine learning model for each pair of exposure and outcome. For example, for Xi exposures and Yj outcomes, we will have Xi times Yj trained models predicting association between respective pairs of outcomes and exposures. The system can train multiple models for each pair of exposure and outcome. For example, we will have multiple trained models for smoking and lung cancer pair, smoking and obesity pair, and so on. Each of the multiple models for the same pair of exposure and outcome can predict a different output. Examples of outputs that can be predicted include accuracy, variance explained, risk, pvalue, false discovery rate, etc. We briefly explain these outputs below.

Accuracy can be defined as the number of correct predictions made by the machine learning model divided by the total number of predictions made, then multiplied by 100 to turn it into a percentage. Accuracy is the number of correctly predicted data points out of all the data points. Often accuracy is used with precision and recall which are other metrics of measures of performance of a machine learning model.

Variance explained (or explained variance) is another output that can be predicted by the trained model. Explained variance is used to measure the discrepancy between a model and actual data. In other words, it is the part of the model's total variance that is explained by factors that are actually present and not due to error variance. Higher percentages of explained variance indicates a stronger strength of association. It also means that model makes better predictions.

Another output from the model is p-value. When we perform a statistical test a p-value helps us determine the significance of results in relation to the null hypothesis. The null hypothesis states that there is no relationship between the two variables being studied (one variable does not affect the other). It states the results are due to chance and are not significant in terms of supporting the idea being investigated. Thus, the null hypothesis assumes that whatever we are trying to prove did not happen. The level of statistical significance is often expressed as a p-value between 0 and 1. The smaller the p-value, the stronger the evidence that we reject the null hypothesis.

The false discovery rate (FDR) is a statistical approach used in multiple hypothesis testing to correct for multiple comparisons. The false discovery rate (FDR) is the expected proportion of type I errors. A type I error is where we incorrectly reject the null hypothesis, in other words, we get a false positive. The false discovery rate is the ratio of the number of false positive results to the number of total positive test results.

FIG. 6B presents a high-level workflow 650 for the X-Y association where X is an exposure (such as dietary intake, smoking, etc.) and Y is an outcome (such as obesity, lung cancer, etc.). The illustration shows that the system can regress Y on X using machine learning model such as LASSO, neural networks, regressors, etc. To reduce the impact of confounders the correlation matrix 351 (labeled as RM in illustration 650) is provided as an additional input to the machine learning model as indicated in part A 652. Using the correlation matrix, the technology disclosed can systematically test every outcome Y against every exposure X while controlling for relatedness between persons. In another implementation (B) labeled as 654 in illustration, the system can provide an additional input Xc which represents a choice of adjustment such as a propensity score.

The illustration 650 also shows examples of outputs (accuracy, variance explained, risk, pvalue, false discovery rate, etc.) from the model which are described above. The system can use a different trained model for each output. For each pair of exposures and outcomes the system can produce all of the outputs from respective trained models.

The technology disclosed includes the logic to evaluate the ranked list of associations between exposures and outcomes to predict risk factors. This process is listed as robustness check (670) in FIG. 6B. The system can perform robustness check by varying the sample size (or population) or by performing vibration of effects by choosing different Xc values. The system can also vary the machine learning models to perform the robustness check.

### Process Flow

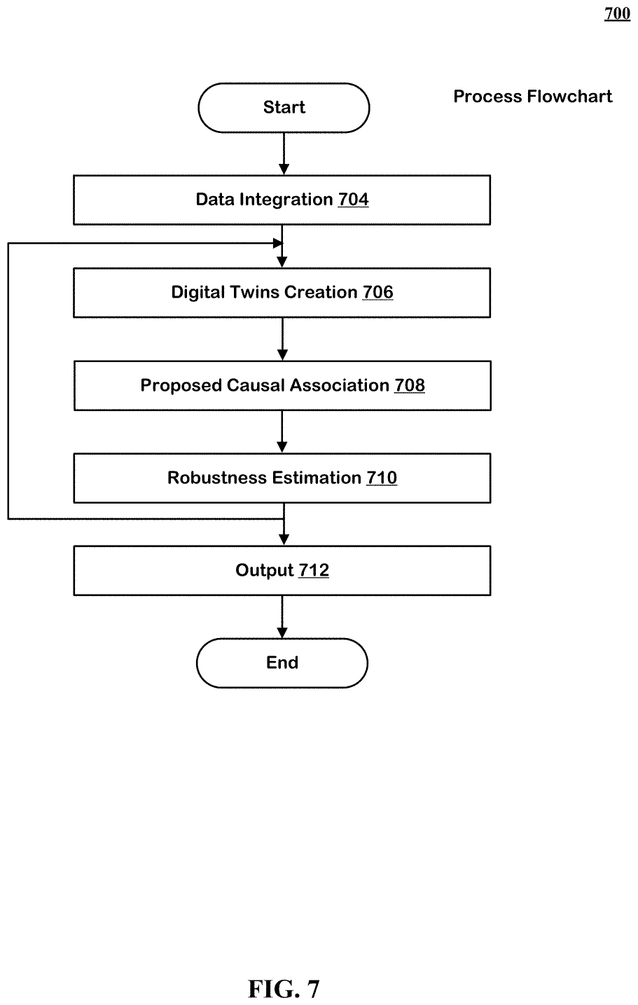

Reference is now made to FIG. 7 which is a flow chart 700 illustrating an example workflow to derive and verify causal association utilizing digital twins. The workflow to derive and verify causal association utilizing digital twins comprises step 704 data integration, step 706 digital twins creation, step 708 proposed causal association, step 710 robustness estimation, and step 712 output. Feedback may also occur after step 710 and go back to step 706. Further reference can be made to FIGS. 6A and 6B, illustrating a workflow to derive and verify causal association utilizing digital twins.

An example input dataset to a digital twins pipeline can be an observational cohort dataset. FIG. 1 shows examples of digital twins pipeline datasets, which comprises insurance claims database with insurance claim data, health record database with digital health record data, personal (or application) database with health or lifestyle related digital data, or patient cohort dataset with any other patient medical data. The digital twins pipeline is configured to integrate these data, cross reference them, or join them with patient information. Patient information can comprise data of a person level, such as a person's identification, data of an area level, such as integrated data of addresses or geographical coordinates, and data of subpopulation level, such as integrated data by a range of value, e.g., a physiological measurement or age group. These data can be described with X-Y coordinates with one axis of the dataset individual at different points in time, and the other axis a time-dependent description of the individual. Further, these data are to be integrated with person or individual-level data and group or area-level data.

Data can be categorized into individual-level and group-level data. These higher-level data categories can include administrative (or administration) data, personal data, area-level or geoexposome data, socioeconomic data, disease prevalence data, and subpopulation data. Administrative data are the central source of information on the health status of an individual as recorded by insurance companies, hospitals (electronic health record), and/or epidemiological cohorts. These data can also include laboratory and physiological measurements, disease history or billing codes. Person level data are data integrated from an individual's applications of the person's mobile devices, for example, APPLE RESEARCHKIT™ applications or applications deployed by contract research organizations (CROs). Data collection at personal level is a potential point of recruitment and consent of individuals and to provide information about individual which is not available in the administrative data and complement the administrative data for a better full picture of an individual's health related information, such as passively recorded information of location, step counts, cardiac rate, etc., and actively recorded information provided by individuals through the interface of application programs.

Thereafter, the set of exposure used as X variables, such as environmental factors, drugs, or other integrated characteristics, and the set of outcomes used as Y variables, such as diagnosed diseases, etc., are identified.

In step 706, a digital twins cohort is created in accordance with the following steps. First, a distance measure between each person in the cohort, such as a Hamming distance, correlation, or other distance measure, is to be defined to create a phenotypic and environmental relatedness matrix 251. The phenotypic and environmental relatedness matrix 251 is conceptually similar to a genetic relatedness matrix. Individuals who are genetic twins have a 0 distance between them, while individuals who are unrelated have a large genetic distance between them. The variables input to measure the distance of two individuals for the phenotypic and environmental relatedness matrix may include without limitation to geographical distance between locations, geographical environmental exposure such as exposure to certain level of air quality, genetic relatedness, e.g., sibling fraternal, identical twins, or fraction of genetic relatedness, phenotypic relatedness based on disease codes or other patient level attribute. By doing so, each individual is a vector, or tensor in another word, with all attributes about the individual and distance value between the individual with another individual representing the two persons' relatedness by the physiological and medical distance between them. The larger the distance value is, the more remote the distance, the less related, and the less likely to be a digital twin to each other. The smaller the distance value is, the less remote the distance, the more related, and the more likely to be a digital twin. Different instantiations of the distance matrix as a function of the parameters, e.g., variables used to estimate distance, the population used, are saved to estimate the sensitivity of the distance which is known as vibration of effects.

Secondly, a machine learning model is trained to predict individuals (or persons) who are most likely to be genetic or phenotypic twins. The targets of such prediction are actual individuals who are genetically closely related to each other. The machine learning algorithm then proceeds to predict the characteristics in the data that are shared between twins. The algorithm is then to be deployed amongst the entire cohort and the distance between individuals is the predicted probability that they are twins. Similarly, different instantiations of the machine learning algorithm of twins as a function of the parameters, e.g., variables used to estimate distance, the population used, are saved to estimate the sensitivity of the distance which is known as vibration of effects.

In some embodiments, cohort can be achieved by building a digital twin which is built by mimicking the physics of a real-world physical object or system. The purpose of a digital object or system is to develop a mathematical model that simulates the real-world original in digital space. The digital twin is constructed to receive inputs from data from a real-world counterpart. Therefore, the digital twin is configured to simulate and offer insights into performance and potential problems of the physical counterpart.

In some implementations, the machine learning model is used to predict the propensity of being exposed to a variable X, i.e., a propensity score. This propensity score estimates the probability of getting an exposure X as a function of all the measured other Xs in the cohort. The output of the machine learning algorithm is the propensity score.

Once a digital twins cohort and the associated set of exposures and outcomes X and Y, respectively, are identified in step 706, in step 708, statistical associations are ascertained between each variable in X and each variable in Y using a regression method. Such regression method can be machine learning algorithms with regression, LASSO with least absolute shrinkage and selection operator, or other machine learning models. The regression model is configured to function in the following operations to incorporate the digital twins.

The environmental and phenotypic correlation matrix 351 identified in step 706 is input as a random effect. By such importation, the correlation accounts for the relatedness between individuals. The binary X can indicate binary exposure in step 706, e.g., smoking. The usual framework for propensity score-based association testing can be employed. Output of step 708 is a ranked list of proposed causal associations. In some implementations, the ranked list of causal associations is for each X-Y pair, e.g., smoking X and cancer Y. In some implementations, the ranked list of causal associations is for a set of Xs and a single Y, e.g., all environmental factors of multiple Xs and asthma Y. Or in some implementations, the ranked list of causal associations is for a set of Xs and a set of Ys, e.g., all environmental factors of multiple Xs and all diseases outcomes of multiple Ys. These associations can be ranked by their summary statistics which may include accuracy, variance explained, risk, odds ratio, pvalue of the prediction or association, etc.

In step 710 robustness of each X-Y association is estimated. The disclosed methods have the merit to search all possible associations between exposures (X/Xs) and outcomes (Y/Ys) and return a ranked list of all possibilities while accounting for relatedness through the digital twin procedure in step 706 to account for confounding. The procedure to automatically evaluate the ranked list and the strongest risk factors involves testing the robustness of the findings by perturbing the analytical study design.

The perturbation of the analytical study design comprises varying the sample size of the digital twins, stratifying the analysis to subsets of the population, e.g., males v. females, covariate selection, etc., or varying the models used in the machine learning algorithm, e.g., regression methods, neural networks, etc. If the rank of an X is robust to such perturbations of the analytic design, the more likely the finding is an association close to the true association between the exposures (X/Xs) and outcomes (Y/Ys). The pipeline can be configured to automatically iterate through combinations of a study design, e.g., analyzing multiple strata of a population, and further test how different estimates are in different strata, or how the risk estimates change. The more the predictions or risk shift as a function of a study design shifts, the less robust the causal association is. For example, using National Health and Nutrition Examination Survey data, vibration of effects, which is a standardized approach to quantify variability of results obtained with choices of adjustments, can be used to demonstrate the instability of observational causal associations. In some embodiments, the results of robustness estimates of associations can be feedback to step 706 for further digital twins creation.

In some embodiments, the pipeline can be configured to integrate other datasets. For example, epidemiological datasets from the National Health and Nutrition Examination Survey, can be integrated to compare risk estimates and meta-analyze risk estimates across cohorts. By doing this, a result from a given cohort can be systematically compared against another cohort.

The disclosed digital twins pipeline can be used to find novel uses for existing drugs, to evaluate risk prediction of environmental factors, to query multiple geographies of disease risk for potential intervention, or to create hypotheses for new interventions in a population.

The disclosed digital twins creation and causal association formation methods create a ranked list of digital twins for large observational, heterogeneous datasets from healthcare systems and personal devices, measure dynamically the similarity between all pairs as a function of parameters based on biomedical data which are used for matching to assess the quality of matching, create a ranked list of all correlations in exposures Xs and outcomes Ys for all elements measured in the cohort database, and further predict digital twins of data that is not directly measured in any individual from integrated sources. Comparing to traditional clinical trials which are designed to examine only one exposure and one outcome at a time, in digital twins platform, all correlations between all putative variables Xs and Ys are associated. Moreover, the disclosed methods can determine the sensitivity of the results to model specification, e.g., confounding or vibration of effects, and to account for multiple comparisons.

The disclosed digital twins creation and causal association formation methods can be applied for hospitals to predict medical health care utilization. It is critical for logistic planning such as appointment arrangements and medical supplies prediction which includes prediction of hospital utilization as a function of other observational data.

The disclosed digital twins creation and causal association formation methods can be applied for estimation of causes and effect of diseases and trends for users such as major health insurance companies and hospitals. Such system comprises a server such as a web application program to display predictions to users. Such system can be configured to enable users to query aggregate health statistics and cause and effect trends for certain regions and individuals.

The disclosed digital twins creation and causal association formation methods can be applied to predict and manage risk and uncertainty in actuarial processes. Health, housing, and life insurances require biomedical data for accurate assessment of risk to optimize pricing of insurance instruments. In such embodiments, each individual is to be mapped to biomedical information to allow actuaries to develop new methods for pricing that is a function of biomedical factors.

The disclosed digital twins creation and causal association formation methods can be also applied to present risk at personal level. In such embodiments, individual disease risk as a function of biomedical factors estimated as a probability can be displayed to end users.

It is to be understood that, because some of the constituent system components and method steps depicted in the accompanying figures can be implemented in Software, the actual connections between the systems components (or the process steps) may differ depending on the fashion in which the present disclosure is programmed. Given the teachings of the present disclosure provided herein, one of ordinary skill in the similar art will be able to contemplate these and similar implementations or configurations of the present disclosure.

It is to be understood that the configuration and boundaries of the functional building blocks of the system have been defined herein for the convenience of the description. Alternative boundaries may be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. While the present disclosure has been described in detail with reference to exemplary embodiments, those skilled in the similar art will appreciate that various modifications and substitutions can be made thereto without departing from the spirit and scope of the disclosure.

### Example of Data Integration

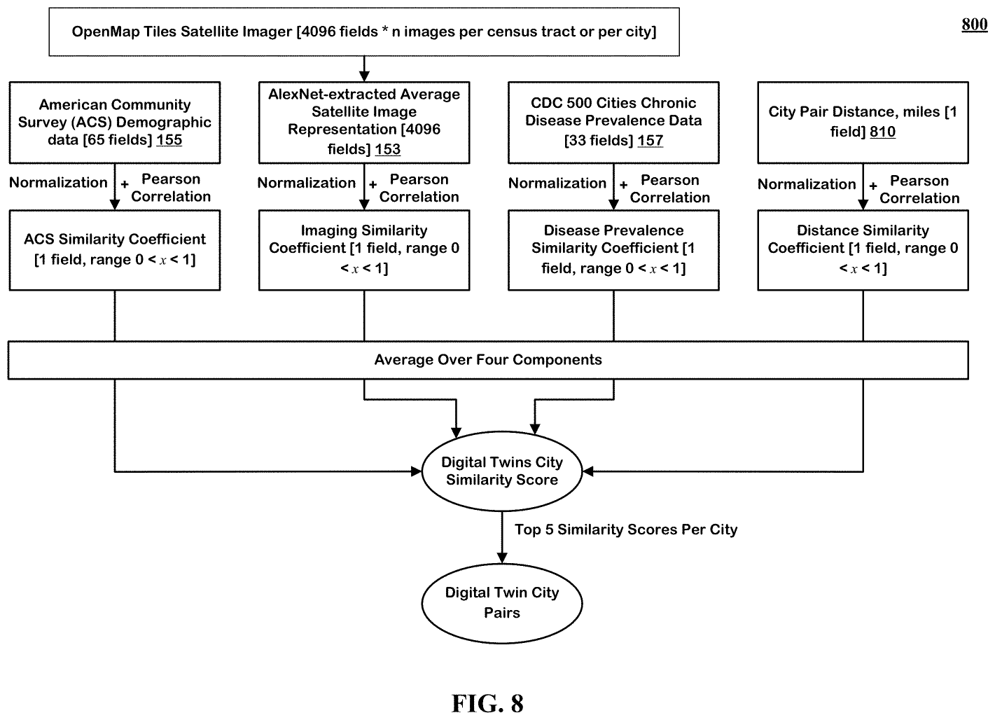

The technology disclosed includes logic to combine data from multiple datasets for predicting digital twins. Data integrator 181 includes data normalizer and data aggregator that can implement the logic to integrate data from multiple observational datasets. FIG. 8 presents an example of integrating data from four different datasets to illustrate the integration process. The illustration 800 shows data from three datasets i.e., socioeconomic database 155, geoexposome database 153, and disease prevalence database 157.

The image data is preprocessed using pretrained machine learning model. The box 153 in FIG. 8 includes extracted features from satellite images. The images can be organized according to a census tract or a city-level geographical area. We passed images through AlexNet, a pretrained convolutional neural network, in an unsupervised deep learning approach called feature extraction. The resulting vector from this process is a “latent space feature” representation of the image comprising 4,096 features. This latent space representation is an encoded (non-human readable) version of the visual patterns found in the satellite images, which, when coupled with machine learning approaches, is used to model the built environment of a given census tract. For each census tract, we calculated the mean of the latent space feature representation.

The fourth input data shown in FIG. 8 is a city pair distance that can indicate the distance between cities, counties, or locations of persons. The number of fields per dataset are also mentioned. For example, dataset 155 includes 65 fields per record, dataset 153 includes 4096 fields, dataset 157 includes 33 fields, and dataset 810 includes 1 field.

For all features (XY, ACS, and CDC), we calculate the weighted average by population to aggregate the data from the census tract level to the city level. Then, all features are standardized to mean 0 and unit variance. In one implementation, the data can be organized at the census tract-level and not aggregated at the city level.

In the example shown in FIG. 8, as the data sources are at the group-level or subpopulation level, the similarity score is calculated between two cities or census tracts. The epidemiological similarity measure is calculated between two cities by taking the average of the four elements: the three correlation coefficients (comprising a holistic view of a city's built environment, demographic factors, and disease prevalence) and the normalized log distance between each city pair.

City groups, or “twins”, are arbitrarily grouped as each city's top 5 most epidemiologically similar cities. All possible city pairs were ranked according to the epidemiological similarity measurement and then the top 5 arbitrarily most similar cities were identified as the Digital City Twins.

The data integration example describe above, can be extended by taking the person-level datasets as input and combining the person-level data with group-level data to predict digital twins of persons in the dataset.

We present examples of machine learning models including their training in the following text. The technology disclosed can use these or similar machine learning models in the digital twins pipeline.

### Examples of Machine Learning Models

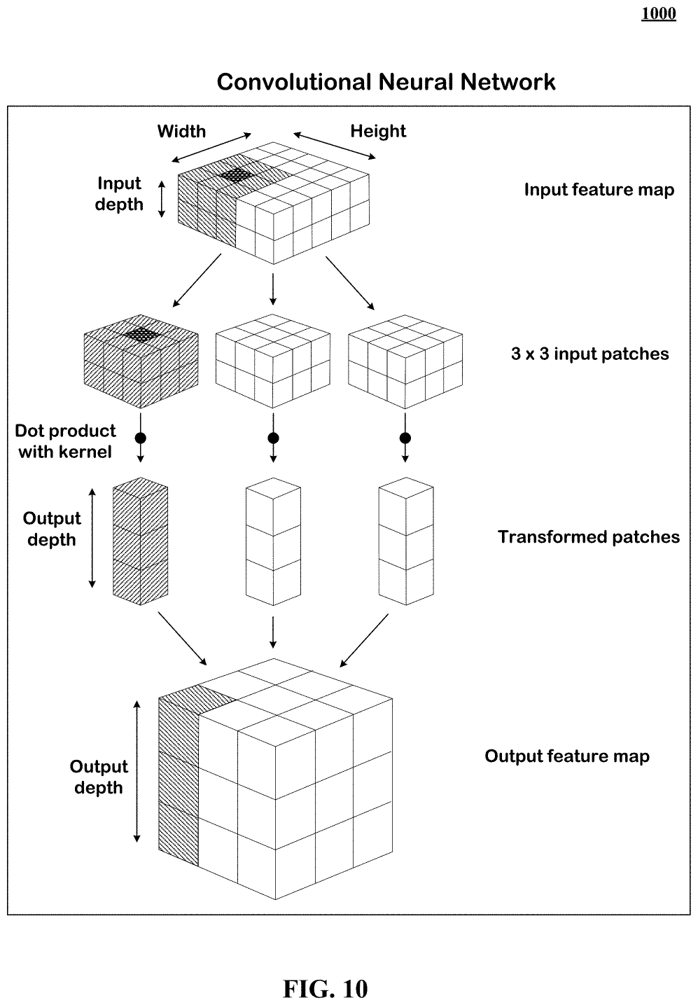

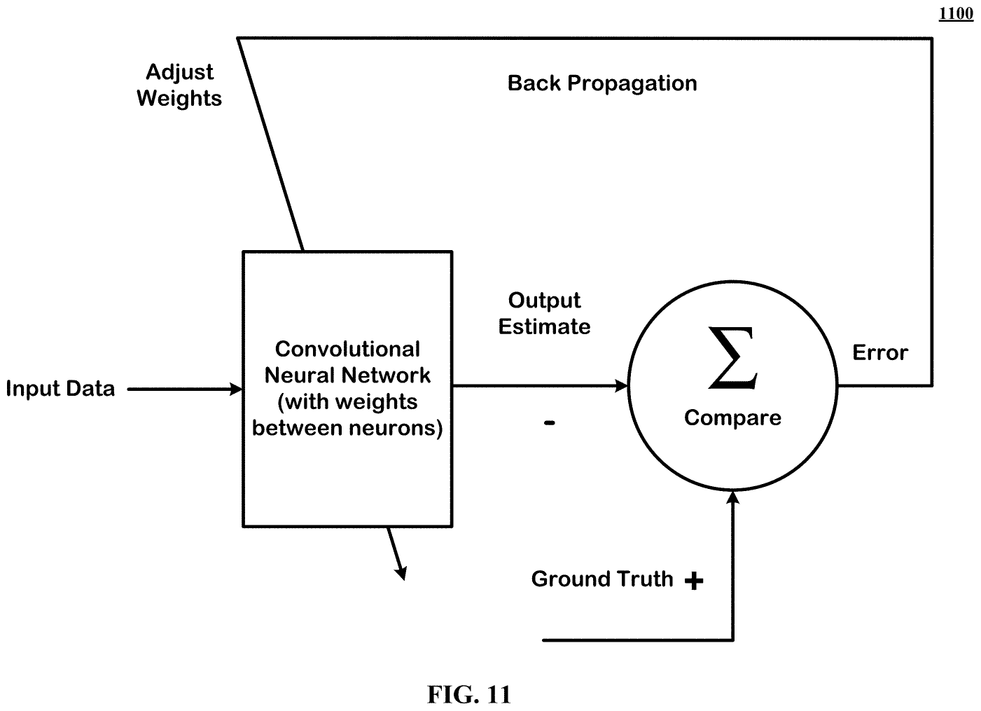

We present a general discussion of random forest machine learning technique as a first example of machine learning models that can be used by the technology disclosed. A general discussion regarding convolutional neural networks, CNNs, and training by gradient descent is presented as a second example of a machine learning model that can be used by the technology disclosed. The discussion of CNNs is facilitated by FIGS. 10-11.

Random Forest Model

Random forest (also referred to as random decision forest) is an ensemble machine learning technique. Ensemble techniques or algorithms combine more than one technique of the same or different kind for classifying objects. The random forest classifier consists of multiple decision trees that operate as an ensemble. Each individual decision tree in random forest acts as a base classifier and outputs a class prediction. The class with the most votes becomes the random forest model's prediction. The fundamental concept behind random forests is that a large number of relatively uncorrelated models (decision trees) operating as a committee will outperform any of the individual constituent models.

Random Forest is an ensemble machine learning technique based on bagging. In bagging-based techniques, during training, subsamples of records are used to train different models such as decision trees in random forest. In addition, feature subsampling can also be used. The idea is that different models will be trained on different types of features and therefore, overall, the model will perform well in production. The output of random forest is based on the output of individual models such as decision trees. The output from individual models is combined to produce the output from the random forest model.

Decision trees are prone to overfitting. To overcome this issue, bagging technique is used to train the decision trees in random forest. Bagging is a combination of bootstrap and aggregation techniques. In bootstrap, during training, we take a sample of rows from our training database and use it to train each decision tree in the random forest. For example, a subset of features for the selected rows can be used in training of decision tree 1. Therefore, the training data for decision tree 1 can be referred to as row sample 1 with column sample 1 or RS1+CS1. The columns or features can be selected randomly. The decision tree 2 and subsequent decision trees in the random forest are trained in a similar manner by using a subset of the training data. Note that the training data for decision trees is generated with replacement i.e., same row data can be used in training of multiple decision trees.

The second part of bagging technique is the aggregation part which is applied during production. Each decision tree outputs a classification for each class. In case of binary classification, it can be 1 or 0. The output of the random forest is the aggregation of outputs of decision trees in the random forest with a majority vote selected as the output of the random forest. By using votes from multiple decision trees, a random forest reduces high variance in results of decision trees, thus resulting in good prediction results. By using row and column sampling to train individual decision trees, each decision tree becomes an expert with respect to training records with selected features.

During training, the output of the random forest is compared with ground truth labels and a prediction error is calculated. During backward propagation, the weights or coefficients of the model are adjusted so that the prediction error is reduced.

CNNs

A convolutional neural network is a special type of neural network. The fundamental difference between a densely connected layer and a convolution layer is this: Dense layers learn global patterns in their input feature space, whereas convolution layers learn local patterns: in the case of images, patterns found in small 2D windows of the inputs. This key characteristic gives convolutional neural networks two interesting properties: (1) the patterns they learn are translation invariant and (2) they can learn spatial hierarchies of patterns. FIG. 10 presents an example convolution neural network 1000.

Regarding the first, after learning a certain pattern in the lower-right corner of a picture, a convolution layer can recognize it anywhere: for example, in the upper-left corner. A densely connected network would have to learn the pattern anew if it appeared at a new location. This makes convolutional neural networks data efficient because they need fewer training samples to learn representations, they have generalization power.

Regarding the second, a first convolution layer can learn small local patterns such as edges, a second convolution layer will learn larger patterns made of the features of the first layers, and so on. This allows convolutional neural networks to efficiently learn increasingly complex and abstract visual concepts.

A convolutional neural network learns highly non-linear mappings by interconnecting layers of artificial neurons arranged in many different layers with activation functions that make the layers dependent. It includes one or more convolutional layers, interspersed with one or more sub-sampling layers and non-linear layers, which are typically followed by one or more fully connected layers. Each element of the convolutional neural network receives inputs from a set of features in the previous layer. The convolutional neural network learns concurrently because the neurons in the same feature map have identical weights. These local shared weights reduce the complexity of the network such that when multi-dimensional input data enters the network, the convolutional neural network avoids the complexity of data reconstruction in feature extraction and regression or classification process.

Convolutions operate over 3D tensors, called feature maps, with two spatial axes (height and width) as well as a depth axis (also called the channels axis). For an RGB image, the dimension of the depth axis is 3, because the image has three color channels; red, green, and blue. For a black-and-white picture, the depth is 1 (levels of gray). The convolution operation extracts patches from its input feature map and applies the same transformation to all of these patches, producing an output feature map. This output feature map is still a 3D tensor: it has a width and a height. Its depth can be arbitrary, because the output depth is a parameter of the layer, and the different channels in that depth axis no longer stand for specific colors as in RGB input; rather, they stand for filters. Filters encode specific aspects of the input data: at a height level, a single filter could encode the concept “presence of a face in the input,” for instance.

For example, the first convolution layer takes a feature map of size (28, 28, 1) and outputs a feature map of size (26, 26, 32): it computes 32 filters over its input. Each of these 32 output channels contains a 26×26 grid of values, which is a response map of the filter over the input, indicating the response of that filter pattern at different locations in the input. That is what the term feature map means: every dimension in the depth axis is a feature (or filter), and the 2D tensor output [:, :, n] is the 2D spatial map of the response of this filter over the input.

Convolutions are defined by two key parameters: (1) size of the patches extracted from the inputs—these are typically 1×1, 3×3 or 5×5 and (2) depth of the output feature map—the number of filters computed by the convolution. Often these start with a depth of 32, continue to a depth of 64, and terminate with a depth of 128 or 256.

A convolution works by sliding these windows of size 3×3 or 5×5 over the 3D input feature map, stopping at every location, and extracting the 3D patch of surrounding features (shape (window_height, window_width, input_depth)). Each such 3D patch is then transformed (via a tensor product with the same learned weight matrix, called the convolution kernel) into a 1D vector of shape (output_depth). All of these vectors are then spatially reassembled into a 3D output map of shape (height, width, output_depth). Every spatial location in the output feature map corresponds to the same location in the input feature map (for example, the lower-right corner of the output contains information about the lower-right corner of the input). For instance, with 3×3 windows, the vector output [i, j, :] comes from the 3D patch input [i−1: i+1, j−1:J+1, :].

The convolutional neural network comprises convolution layers which perform the convolution operation between the input values and convolution filters (matrix of weights) that are learned over many gradient update iterations during the training. Let (m, n) be the filter size and W be the matrix of weights, then a convolution layer performs a convolution of the W with the input X by calculating the dot product W·x+b, where x is an instance of X and b is the bias. The step size by which the convolution filters slide across the input is called the stride, and the filter area (m×n) is called the receptive field. A same convolution filter is applied across different positions of the input, which reduces the number of weights learned. It also allows location invariant learning, i.e., if an important pattern exists in the input, the convolution filters learn it no matter where it is in the sequence.

Training a Convolutional Neural Network

FIG. 11 depicts a block diagram 1100 of training a convolutional neural network in accordance with one implementation of the technology disclosed. The convolutional neural network is adjusted or trained so that the input data leads to a specific output estimate. The convolutional neural network is adjusted using back propagation based on a comparison of the output estimate and the ground truth until the output estimate progressively matches or approaches the ground truth.

The convolutional neural network is trained by adjusting the weights between the neurons based on the difference between the ground truth and the actual output. This is mathematically described as:

Δwᵢ=xᵢδ
where δ=(ground truth)−(actual output)

In one implementation, the training rule is defined as:

Wₙₘ←Wₙₘ+α(tₘ−φₘ)aₙ

In the equation above: the arrow indicates an update of the value; tₘ is the target value of neuron m; φₘ is the computed current output of neuron m; aₙ is input n; and α is the learning rate.

The intermediary step in the training includes generating a feature vector from the input data using the convolution layers. The gradient with respect to the weights in each layer, starting at the output, is calculated. This is referred to as the backward pass, or going backwards. The weights in the network are updated using a combination of the negative gradient and previous weights.

In one implementation, the convolutional neural network uses a stochastic gradient update algorithm (such as ADAM) that performs backward propagation of errors by means of gradient descent. One example of a sigmoid function based back propagation algorithm is described below:

$$ϕ = f ( h ) = \frac{1}{1 + {e}^{- h}}$$

In the sigmoid function above, h is the weighted sum computed by a neuron. The sigmoid function has the following derivative:

$$\frac{\partial ϕ}{\partial h} = ϕ ( 1 - ϕ )$$

The algorithm includes computing the activation of all neurons in the network, yielding an output for the forward pass. The activation of neuron m in the hidden layers is described as:

$${ϕ}_{m} = \frac{1}{1 + {e}^{- hm}}$$

$${h}_{m} = {\sum }_{n = 1}^{N} {a}_{n} {w}_{nm}$$

This is done for all the hidden layers to get the activation described as:

$${ϕ}_{k} = \frac{1}{1 + {e}^{{h}_{k}}}$$

$${h}_{k} = {\sum }_{m = 1}^{M} {ϕ}_{m} {v}_{mk}$$

Then, the error and the correct weights are calculated per layer. The error at the output is computed as:

δₒₖ=(tₖ−φₖ)φₖ(1−φₖ)

The error in the hidden layers is calculated as:

$${δ}_{hm} = {ϕ}_{m} ( 1 - {ϕ}_{m} ) {\sum }_{k = 1}^{K} {v}_{mk} {δ}_{ok}$$

The weights of the output layer are updated as:

νmk←νmk+αδokφm

The weights of the hidden layers are updated using the learning rate α as:

νnm←wnm+αθhman

In one implementation, the convolutional neural network uses a gradient descent optimization to compute the error across all the layers. In such an optimization, for an input feature vector x and the predicted output ŷ, the loss function is defined as l for the cost of predicting ŷ when the target is y, i.e., l(ŷ, y). The predicted output ŷ is transformed from the input feature vector x using function ƒ. Function ƒ is parameterized by the weights of convolutional neural network, i.e., ŷ=ƒ_{w}(x). The loss function is described as l(ŷ, y)=l(ƒ_{w}(x), y), or

Q(z, w)=l(ƒ_{w}(x), y) where z is an input and output data pair (x, y). The gradient descent optimization is performed by updating the weights according to:

$${v}_{t + 1} = μ {v}_{t} - α \frac{1}{n} {\sum }_{i = 1}^{N} ∇ {w}_{t} Q ( {z}_{t} , {w}_{t} )$$

$${w}_{t + 1} = {w}_{t} + {v}_{t + 1}$$

In the equations above, α is the learning rate. Also, the loss is computed as the average over a set of n data pairs. The computation is terminated when the learning rate α is small enough upon linear convergence. In other implementations, the gradient is calculated using only selected data pairs fed to a Nesterov's accelerated gradient and an adaptive gradient to inject computation efficiency.

In one implementation, the convolutional neural network uses a stochastic gradient descent (SGD) to calculate the cost function. An SGD approximates the gradient with respect to the weights in the loss function by computing it from only one, randomized, data pair, zₜ, described as:

νₜ₊₁=μν−α∇wQ(zₜ,wₜ)

wₜ₊₁=wₜ+νₜ₊₁

In the equations above: α is the learning rate; μ is the momentum; and t is the current weight state before updating. The convergence speed of SGD is approximately O(1/t) when the learning rate α are reduced both fast and slow enough. In other implementations, the convolutional neural network uses different loss functions such as Euclidean loss and softmax loss. In a further implementation, an ADAM stochastic optimizer is used by the convolutional neural network.

### Particular Implementations

We describe implementations of a system for predicting digital twins and using the prediction in determining causal relationships between exposures and outcomes.

The technology disclosed can be practiced as a system, method, or article of manufacture. One or more features of an implementation can be combined with the base implementation. Implementations that are not mutually exclusive are taught to be combinable. One or more features of an implementation can be combined with other implementations. This disclosure periodically reminds the user of these options. Omission from some implementations of recitations that repeat these options should not be taken as limiting the combinations taught in the preceding sections—these recitations are hereby incorporated forward by reference into each of the following implementations.

A first system implementation of the technology disclosed includes one or more processors coupled to memory. The memory can be loaded with instructions to predict digital twins. The system can include logic to determine a correlation value for a first person indicating a distance of the first person from a second person in the plurality of persons using inputs from one or more types of observational datasets. The system can include a trained regressor. The inputs to the regressor can be from one or more of the following types of datasets.

A first individual-level administration dataset can include clinical data of respective health statuses of the first person and the second person. The administration dataset can include disease codes (ICD), drug codes (NCD), procedure codes (CPT), billing codes, and physiological measurements, etc. A second individual-level person dataset can include personal data of respective health statuses of the first person and the second person. The personal dataset can include passively recorded data from the first person and the second person including location, step count, heart rate. The personal dataset can also include actively recorded data from the first person and the second person including height, weight, and images of prescription drugs. A third group-level exposome dataset can include environmental exposure of the first person and the second person using their respective geographical location. The third group-level exposome dataset can comprise a geoexposome image dataset, a demographic and socioeconomic factors dataset, and a disease prevalence dataset. The geoexposome image dataset can include satellite image data of built environment per census tract and geographical sensor-based data per census-tract. The demographic and socioeconomic factors dataset can include ethnicity, income indicators, education indicators, housing indicators, health insurance type, age, occupations per census tract, etc. The disease prevalence dataset can include disease prevalence information per census tract. A fourth group-level subpopulation dataset can include age-range and laboratory-range characteristics.

The system includes logic to output the correlation value from the trained regressor. The correlation value can indicate distance of the first person from the second person in the plurality of persons. The system can compare the correlation value with a threshold. The system includes logic to report the correlation value in an environmental and phenotypic correlation matrix listing persons in the plurality of persons along rows and columns. The correlation value indicates the first person as a digital twin of the second person in the plurality of persons when the correlation value is above the threshold.

This system implementation and other systems disclosed optionally include one or more of the following features. System can also include features described in connection with methods disclosed. In the interest of conciseness, alternative combinations of system features are not individually enumerated. Features applicable to systems, methods, and articles of manufacture are not repeated for each statutory class set of base features. The reader will understand how features identified in this section can readily be combined with base features in other statutory classes.

In one implementation, the system includes logic to determine a ranked list of causal relationships between a plurality of exposures and a plurality of outcomes. The system includes logic to determine this causal relationship by systemically iterating for each exposure in the plurality of exposures and each outcome in the plurality of outcomes. The system includes logic to provide, in each iteration, to a second trained regressor, a pair of exposure and outcome from the plurality of exposures and the plurality of outcomes and the environmental and phenotypic correlation matrix as inputs. The system includes logic to predict an association value for the pair of exposure and outcome from the second trained regressor. The system includes logic to report the association value for the pair of exposure and outcome in a ranked list of causal relationships between exposures in the plurality of exposures and outcomes in the plurality of outcomes.

The data in the observational datasets can be encoded with temporal data including time series metrics over a given time period.

The exposure in the pair of exposure and outcome is dietary intake and the outcome in the pair of exposure and outcome is obesity.

The exposure in the pair of exposure and outcome is dietary intake and the outcome in the pair of exposure and outcome is diabetes.

The exposure in the pair of exposure and outcome is smoking and the outcome in the pair of exposure and outcome is lung cancer.

Other implementations consistent with this system may include a non-transitory computer readable storage medium storing instructions executable by a processor to perform functions of the system described above. Yet another implementation may include a method performing the functions of the system described above.

A second system implementation of the technology disclosed includes one or more processors coupled to memory. The memory can be loaded with instructions to predict digital twins. The system can include logic to determine a correlation value for a first person indicating a distance of the first person from a second person in the plurality of persons using inputs from an individual-level dataset and a group-level dataset. The system can use a trained machine learning model such as a regressor to determine the correlation value. The individual-level dataset can include administration data including disease codes (ICD), drug codes (NCD), procedure codes (CPT), billing codes, and physiological measurements. The group-level dataset can include exposome data comprising a geoexposome image dataset, a demographic and socioeconomic factors dataset, and a disease prevalence dataset. The geoexposome image dataset can include satellite image data of built environment per census tract and geographical sensor-based data per census-tract. The demographic and socioeconomic factors dataset can include ethnicity, income indicators, education indicators, housing indicators, health insurance type, age, occupations per census tract. The disease prevalence dataset can include disease prevalence information per census tract. The trained regressor outputs the correlation value indicating distance between the first person and the second person in the plurality of persons and comparing the correlation value with a threshold. The system include logic to report the correlation value in an environmental and phenotypic correlation matrix listing persons in the plurality of persons along rows and columns. The correlation value can indicate the first person as a digital twin of the second person in the plurality of persons when the correlation value is above the threshold.

This system implementation and other systems disclosed optionally include one or more of the following features. System can also include features described in connection with methods disclosed. In the interest of conciseness, alternative combinations of system features are not individually enumerated. Features applicable to systems, methods, and articles of manufacture are not repeated for each statutory class set of base features. The reader will understand how features identified in this section can readily be combined with base features in other statutory classes.

In one implementation, the system can include logic to receive input from an individual-level person dataset. The individual-level person dataset can include personal data of respective health statuses of the first person and the second person. The personal dataset can include passively recorded data from the first person and the second person including location, step count, heart rate. The personal dataset can also include actively recorded data from the first person and the second person including height, weight, and images of prescription drugs.

In one implementation, the system can include logic to receive input from a group-level subpopulation dataset. The group-level subpopulation dataset including age-range and laboratory-range characteristics.

Other implementations consistent with this system may include a non-transitory computer readable storage medium storing instructions executable by a processor to perform functions of the system described above. Yet another implementation may include a method performing the functions of the system described above.

Aspects of the technology disclosed can be practiced as a first method of predicting digital twins. The method can include determining a correlation value for a first person indicating a distance of the first person from a second person in the plurality of persons using inputs from one or more types of observational datasets. The method can include using a trained regressor. The inputs to the regressor can be from one or more of the following types of datasets.

A first individual-level administration dataset can include clinical data of respective health statuses of the first person and the second person. The administration dataset can include disease codes (ICD), drug codes (NCD), procedure codes (CPT), billing codes, and physiological measurements, etc. A second individual-level person dataset can include personal data of respective health statuses of the first person and the second person. The personal dataset can include passively recorded data from the first person and the second person including location, step count, heart rate. The personal dataset can also include actively recorded data from the first person and the second person including height, weight, and images of prescription drugs. A third group-level exposome dataset can include environmental exposure of the first person and the second person using their respective geographical location. The third group-level exposome dataset can comprise a geoexposome image dataset, a demographic and socioeconomic factors dataset, and a disease prevalence dataset. The geoexposome image dataset can include satellite image data of built environment per census tract and geographical sensor-based data per census-tract. The demographic and socioeconomic factors dataset can include ethnicity, income indicators, education indicators, housing indicators, health insurance type, age, occupations per census tract, etc. The disease prevalence dataset can include disease prevalence information per census tract. A fourth group-level subpopulation dataset can include age-range and laboratory-range characteristics.

The method includes outputting the correlation value from the trained regressor. The correlation value can indicate distance of the first person from the second person in the plurality of persons. The method can include comparing the correlation value with a threshold. The method can include reporting the correlation value in an environmental and phenotypic correlation matrix listing persons in the plurality of persons along rows and columns. The correlation value indicates the first person as a digital twin of the second person in the plurality of persons when the correlation value is above the threshold.

This method implementation can incorporate any of the features of the systems described above or throughout this application that apply to the method implemented by the systems. In the interest of conciseness, alternative combinations of method features are not individually enumerated. Features applicable to systems, methods, and articles of manufacture are not repeated for each statutory class set of base features. The reader will understand how features identified in this section for one statutory class can readily be combined with base features in other statutory classes.

Other implementations consistent with this method may include a non-transitory computer readable storage medium storing instructions executable by a processor to perform any of the methods described above. Yet another implementation may include a system with memory loaded from a computer readable storage medium with program instructions to perform the method described above. The system can be loaded from either a transitory or a non-transitory computer readable storage medium.

Aspects of the technology disclosed can be practiced as a second method of predicting digital twins. The second method can include determining a correlation value for a first person indicating a distance of the first person from a second person in the plurality of persons using inputs from an individual-level dataset and a group-level dataset. The method includes using a trained machine learning model such as a regressor to determine the correlation value. The individual-level dataset can include administration data including disease codes (ICD), drug codes (NCD), procedure codes (CPT), billing codes, and physiological measurements. The group-level dataset can include exposome data comprising a geoexposome image dataset, a demographic and socioeconomic factors dataset, and a disease prevalence dataset. The geoexposome image dataset can include satellite image data of built environment per census tract and geographical sensor-based data per census-tract. The demographic and socioeconomic factors dataset can include ethnicity, income indicators, education indicators, housing indicators, health insurance type, age, occupations per census tract. The disease prevalence dataset can include disease prevalence information per census tract. The trained regressor outputs the correlation value indicating distance between the first person and the second person in the plurality of persons and comparing the correlation value with a threshold. The method includes reporting the correlation value in an environmental and phenotypic correlation matrix listing persons in the plurality of persons along rows and columns. The correlation value can indicate the first person as a digital twin of the second person in the plurality of persons when the correlation value is above the threshold.

This method implementation can incorporate any of the features of the systems described above or throughout this application that apply to the method implemented by the system. In the interest of conciseness, alternative combinations of method features are not individually enumerated. Features applicable to systems, methods, and articles of manufacture are not repeated for each statutory class set of base features. The reader will understand how features identified in this section for one statutory class can readily be combined with base features in other statutory classes.

Other implementations consistent with this method may include a non-transitory computer readable storage medium storing instructions executable by a processor to perform any of the methods described above. Yet another implementation may include a system with memory loaded from a computer readable storage medium with program instructions to perform the method described above. The system can be loaded from either a transitory or a non-transitory computer readable storage medium.

As an article of manufacture, rather than a method, a non-transitory computer readable medium (CRM) can be loaded with program instructions executable by a processor. The program instructions when executed, implement the computer-implemented methods described above. Alternatively, the program instructions can be loaded on a non-transitory CRM and, when combined with appropriate hardware, become a component of one or more of the computer-implemented systems that practice the method disclosed.

Each of the features discussed in this particular implementation section for the methods implementation apply equally to CRM implementation. As indicated above, all the method features are not repeated here, in the interest of conciseness, and should be considered repeated by reference.

### Computer System

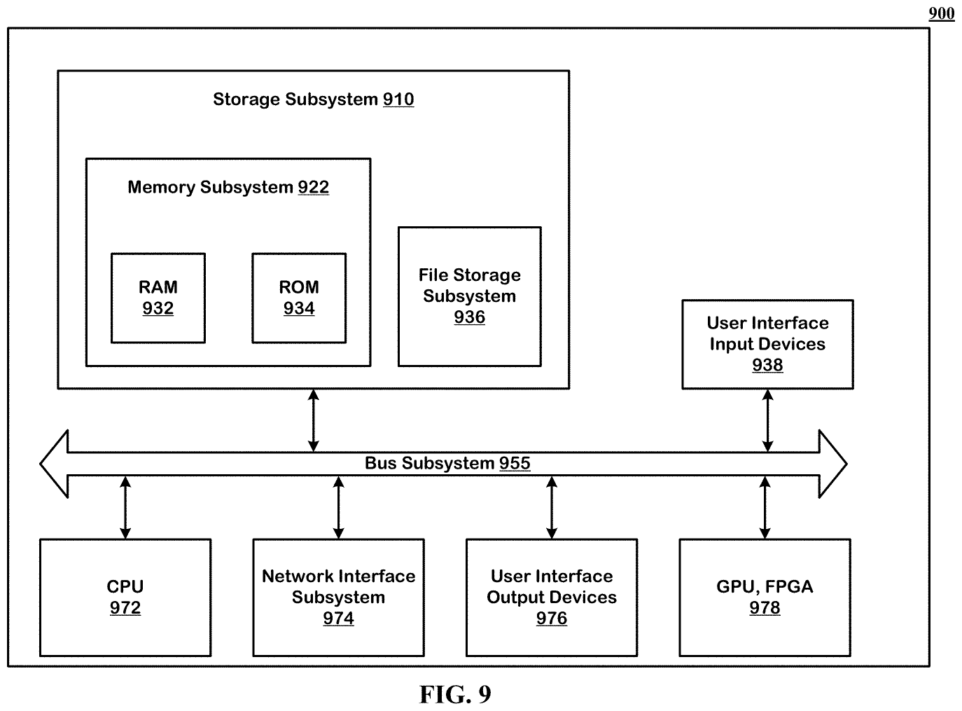

FIG. 9 is a simplified block diagram of a computer system 900 that can be used to implement the technology disclosed. Computer system typically includes at least one processor 972 that communicates with a number of peripheral devices via bus subsystem 955. These peripheral devices can include a storage subsystem 910 including, for example, memory subsystem 922 and a file storage subsystem 936, user interface input devices 938, user interface output devices 976, and a network interface subsystem 974. The input and output devices allow user interaction with computer system. Network interface subsystem provides an interface to outside networks, including an interface to corresponding interface devices in other computer systems.

User interface input devices 938 can include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system.

User interface output devices 976 can include a display subsystem, a printer, a fax machine, or non-visual displays such as audio output devices. The display subsystem can include a cathode ray tube (CRT), a flat-panel device such as a liquid crystal display (LCD), a projection device, or some other mechanism for creating a visible image. The display subsystem can also provide a non-visual display such as audio output devices. In general, use of the term “output device” is intended to include all possible types of devices and ways to output information from computer system to the user or to another machine or computer system.

Storage subsystem 910 stores programming and data constructs that provide the functionality of some or all of the modules and methods described herein. These software modules are generally executed by processor alone or in combination with other processors.

Memory used in the storage subsystem can include a number of memories including a main random access memory (RAM) 932 for storage of instructions and data during program execution and a read only memory (ROM) 934 in which fixed instructions are stored. The file storage subsystem 936 can provide persistent storage for program and data files, and can include a hard disk drive, a floppy disk drive along with associated removable media, a CD-ROM drive, an optical drive, or removable media cartridges. The modules implementing the functionality of certain implementations can be stored by file storage subsystem in the storage subsystem, or in other machines accessible by the processor.

Bus subsystem 955 provides a mechanism for letting the various components and subsystems of computer system communicate with each other as intended. Although bus subsystem is shown schematically as a single bus, alternative implementations of the bus subsystem can use multiple busses.

Computer system itself can be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the description of computer system depicted in FIG. 9 is intended only as a specific example for purposes of illustrating the technology disclosed. Many other configurations of computer system are possible having more or less components than the computer system depicted in FIG. 9.

The computer system 900 includes GPUs or FPGAs 978. It can also include machine learning processors hosted by machine learning cloud platforms such as Google Cloud Platform, Xilinx, and Cirrascale. Examples of deep learning processors include Google's Tensor Processing Unit (TPU), rackmount solutions like GX4 Rackmount Series, GX8 Rackmount Series, NVIDIA DGX-1, Microsoft' Stratix V FPGA, Graphcore's Intelligent Processor Unit (IPU), Qualcomm's Zeroth platform with Snapdragon processors, NVIDIA's Volta, NVIDIA's DRIVE PX, NVIDIA's JETSON TX1/TX2 MODULE, Intel's Nirvana, Movidius VPU, Fujitsu DPI, ARM's DynamiclQ, IBM TrueNorth, and others.

We claim as follows:

1. An artificial intelligence-implemented method of predicting digital twins, including:
determining, for a first person in a plurality of persons, a correlation value indicating a distance of the first person from a second person in the plurality of persons using inputs from two or more types of observational datasets, using a trained regressor; wherein the inputs are from the types of observational datasets that include:
a first individual-level administration dataset including clinical data of respective health statuses of the first person and the second person, the administration dataset including disease codes (ICD), drug codes (NCD), procedure codes (CPT), billing codes, and physiological measurements,
a second individual-level person dataset including personal data of respective health statuses of the first person and the second person, the personal dataset including passively recorded data from the first person and the second person including location, step count, heart rate and actively recorded data from the first person and the second person including height, weight, and images of prescription drugs,
a third group-level exposome dataset including environmental exposure of the first person and the second person using their respective geographical location, the third group-level exposome dataset further comprising a geoexposome image dataset, a demographic and socioeconomic factors dataset, a disease prevalence dataset, wherein,
the geoexposome image dataset including satellite image data of built environment per census tract and geographical sensor-based data per census-tract,
the demographic and socioeconomic factors dataset including ethnicity, income indicators, education indicators, housing indicators, health insurance type, age, occupations per census tract, and
the disease prevalence dataset including disease prevalence information per census tract, and
a fourth group-level subpopulation dataset including age-range and laboratory-range characteristics;
outputting, from the trained regressor, the correlation value indicating distance of the first person from the second person in the plurality of persons and comparing the correlation value with a threshold; and
reporting, in an environmental and phenotypic correlation matrix listing persons in the plurality of persons along rows and columns, the correlation value indicating the first person as a digital twin of the second person in the plurality of persons when the correlation value is above the threshold.

2. The method of claim 1, further including:
determining, between a plurality of exposures and a plurality of outcomes, a ranked list of causal relationships by systemically iterating for each exposure in the plurality of exposures and each outcome in the plurality of outcomes;
providing, in each iteration, to a second trained regressor, a pair of exposure and outcome from the plurality of exposures and the plurality of outcomes and the environmental and phenotypic correlation matrix;
predicting, from the second trained regressor, an association value for the pair of exposure and outcome; and
reporting, in a ranked list of causal relationships between exposures in the plurality of exposures and outcomes in the plurality of outcomes, the association value for the pair of exposure and outcome.

3. The method of claim 1, wherein the data in the observational datasets is encoded with temporal data including time series metrics over a given time period.

4. The method of claim 2, wherein the exposure in the pair of exposure and outcome is dietary intake and the outcome in the pair of exposure and outcome is obesity.

5. The method of claim 2, wherein the exposure in the pair of exposure and outcome is dietary intake and the outcome in the pair of exposure and outcome is diabetes.

6. The method of claim 2, wherein the exposure in the pair of exposure and outcome is smoking and the outcome in the pair of exposure and outcome is lung cancer.

7. A method of predicting digital twins, including:
determining, for a first person in a plurality of persons, a correlation value indicating a distance of the first person from a second person in the plurality of persons using inputs from an individual-level dataset and a group-level dataset, using a trained regressor; wherein:
the individual-level dataset includes administration data including disease codes (ICD), drug codes (NCD), procedure codes (CPT), billing codes, and physiological measurements,
the group-level dataset includes exposome data comprising a geoexposome image dataset, a demographic and socioeconomic factors dataset, and a disease prevalence dataset, wherein,
the geoexposome image dataset including satellite image data of built environment per census tract and geographical sensor-based data per census-tract,
the demographic and socioeconomic factors dataset including ethnicity, income indicators, education indicators, housing indicators, health insurance type, age, occupations per census tract, and
the disease prevalence dataset including disease prevalence information per census tract, and
outputting, from the trained regressor, the correlation value indicating distance between the first person and the second person in the plurality of persons and comparing the correlation value with a threshold; and
reporting, in an environmental and phenotypic correlation matrix listing persons in the plurality of persons along rows and columns, the correlation value indicating the first person as a digital twin of the second person in the plurality of persons when the correlation value is above the threshold.

8. The method of claim 7, further including input from an individual-level person dataset, wherein,
the individual-level person dataset including personal data of respective health statuses of the first person and the second person, the personal dataset including passively recorded data from the first person and the second person including location, step count, heart rate and actively recorded data from the first person and the second person including height, weight, and images of prescription drugs.

9. The method of claim 7, further including input from a group-level subpopulation dataset, wherein,
the group-level subpopulation dataset including age-range and laboratory-range characteristics.

10. A non-transitory computer readable storage medium impressed with computer program instructions to predict digital twins, the instructions, when executed on a processor, implement a method comprising:
determining, for a first person in a plurality of persons, a correlation value indicating a distance of the first person from a second person in the plurality of persons using inputs from two or more types of observational datasets, using a trained regressor; wherein the inputs are from the types of observational datasets that include:
a first individual-level administration dataset including clinical data of respective health statuses of the first person and the second person, the administration dataset including disease codes (ICD), drug codes (NCD), procedure codes (CPT), billing codes, and physiological measurements,
a second individual-level person dataset including personal data of respective health statuses of the first person and the second person, the personal dataset including passively recorded data from the first person and the second person including location, step count, heart rate and actively recorded data from the first person and the second person including height, weight, and images of prescription drugs,
a third group-level exposome dataset including environmental exposure of the first person and the second person using their respective geographical location, the third group-level exposome dataset further comprising a geoexposome image dataset, a demographic and socioeconomic factors dataset, a disease prevalence dataset, wherein,
the geoexposome image dataset including satellite image data of built environment per census tract and geographical sensor-based data per census-tract,
the demographic and socioeconomic factors dataset including ethnicity, income indicators, education indicators, housing indicators, health insurance type, age, occupations per census tract, and
the disease prevalence dataset including disease prevalence information per census tract, and
a fourth group-level subpopulation dataset including age-range and laboratory-range characteristics;
outputting, from the trained regressor, the correlation value indicating distance of the first person from the second person in the plurality of persons and comparing the correlation value with a threshold; and
reporting, in an environmental and phenotypic correlation matrix listing persons in the plurality of persons along rows and columns, the correlation value indicating the first person as a digital twin of the second person in the plurality of persons when the correlation value is above the threshold.

11. The non-transitory computer readable storage medium of claim 10, implementing the method further comprising:
determining, between an exposure in a plurality of exposures and an outcome in a plurality of outcomes, a ranked list of causal relationships by systemically iterating for each exposure in the plurality of exposures and each outcome in the plurality of outcomes;
providing, in each iteration, to a second trained regressor, a pair of exposure and outcome from the plurality of exposures and the plurality of outcomes and the environmental and phenotypic correlation matrix;
predicting, from the second trained regressor, an association value for the pair of exposure and outcome; and
reporting, in a ranked list of causal relationships between exposures in the plurality of exposures and outcomes in the plurality of outcomes, the association value for the pair of exposure and outcome.

12. The non-transitory computer readable storage medium of claim 10, wherein the data in the observational datasets is encoded with temporal data including time series metrics over a given time period.

13. The non-transitory computer readable storage medium of claim 11, wherein the exposure in the pair of exposure and outcome is dietary intake and the outcome in the pair of exposure and outcome is obesity.

14. The non-transitory computer readable storage medium of claim 11, wherein the exposure in the pair of exposure and outcome is dietary intake and the outcome in the pair of exposure and outcome is diabetes.

15. The non-transitory computer readable storage medium of claim 11, wherein the exposure in the pair of exposure and outcome is smoking and the outcome in the pair of exposure and outcome is lung cancer.

16. A system including one or more processors coupled to memory, the memory loaded with computer instructions to predict digital twins, when executed on the processors implement the instructions of claim 10.

17. The system of claim 16, further implementing actions comprising:
determining, between an exposure in a plurality of exposures and an outcome in a plurality of outcomes, a ranked list of causal relationships by systemically iterating for each exposure in the plurality of exposures and each outcome in the plurality of outcomes;
providing, in each iteration, to a second trained regressor, a pair of exposure and outcome from the plurality of exposures and the plurality of outcomes and the environmental and phenotypic correlation matrix;
predicting, from the second trained regressor, an association value for the pair of exposure and outcome, and
reporting, in a ranked list of causal relationships between exposures in the plurality of exposures and outcomes in the plurality of outcomes, the association value for the pair of exposure and outcome.

18. The system of claim 16, wherein the data in the observational datasets is encoded with temporal data including time series metrics over a given time period.

19. The system of claim 17, wherein the exposure in the pair of exposure and outcome is dietary intake and the outcome in the pair of exposure and outcome is obesity.

20. The system of claim 17, wherein the exposure in the pair of exposure and outcome is dietary intake and the outcome in the pair of exposure and outcome is diabetes.
